(19) United States
(12) Patent Application Publication
(10) Pub. No.: US 2020/0291008 A1
(43) Pub. Date: Sep. 17, 2020

(54) MODULATORS OF INDOLEAMINE 2,3-DIOXYGENASE

(71) Applicant: GLAXOSMITHKLINE INTELLECTUAL PROPERTY DEVELOPMENT LIMITED, Brentford, Middlesex (GB)

(72) Inventors: Wieslaw M. KAZMIERSKI, Research Triangle Park, NC (US); John G. CATALANO, Research Triangle Park, NC (US); Pek Y. CHONG, Research Triangle Park, NC (US)

(21) Appl. No.: 16/767,236

(22) PCT Filed: Nov. 28, 2018
(86) PCT No.: PCT/IB2018/059408

§ 371 Date: May 27, 2020

Related U.S. Application Data
(60) Provisional application No. 62/594,724, filed on Dec. 5, 2017.

(51) Int. Cl. C07D 413/08; C07D 215/14; C07D 401/08

(52) U.S. Cl. CPC C07D 413/08; C07D 401/08; C07D 215/14

(57) ABSTRACT

Provided are IDO1 inhibitor compounds of Formula I and pharmaceutically acceptable salts thereof, their pharmaceutical compositions, their methods of preparation, and methods for their use in the prevention and/or treatment of diseases.

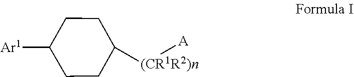

### FIELD OF THE INVENTION

Compounds, methods and pharmaceutical compositions for the prevention and/or treatment of HIV; including the prevention of the progression of AIDS and general immunosuppression, by administering certain indoleamine 2,3-dioxygenase compounds in therapeutically effective amounts are disclosed. Methods for preparing such compounds and methods of using the compounds and pharmaceutical compositions thereof are also disclosed.

### BACKGROUND OF THE INVENTION

Indoleamine-2,3-dioxygenase 1 (IDO1) is a heme-containing enzyme that catalyzes the oxidation of the indole ring of tryptophan to produce N-formyl kynurenine, which is rapidly and constitutively converted to kynurenine (Kyn) and a series of downstream metabolites. IDO1 is the rate limiting step of this kynurenine pathway of tryptophan metabolism and expression of IDO1 is inducible in the context of inflammation. Stimuli that induce IDO1 include viral or bacterial products, or inflammatory cytokines associated with infection, tumors, or sterile tissue damage. Kyn and several downstream metabolites are immunosuppressive: Kyn is antiproliferative and proapoptotic to T cells and NK cells (Munn, Shafizadeh et al. 1999, Frumento, Rotondo et al. 2002) while metabolites such as 3-hydroxy anthranilic acid (3-HAA) or the 3-HAA oxidative dimerization product cinnabarinic acid (CA) inhibit phagocyte function (Sekkai, Guittet et al. 1997), and induce the differentiation of immunosuppressive regulatory T cells (Treg) while inhibiting the differentiation of gut-protective IL-17 or IL-22-producing CD4⁺ T cells (Th17 and Th22)(Favre, Mold et al. 2010). IDO1 induction, among other mechanisms, is likely important in limiting immunopathology during active immune responses, in promoting the resolution of immune responses, and in promoting fetal tolerance. However, in chronic settings, such as cancer, or chronic viral or bacterial infection, IDO1 activity prevents clearance of tumor or pathogen and if activity is systemic, IDO1 activity may result in systemic immune dysfunction (Boasso and Shearer 2008, Li, Huang et al. 2012). In addition to these immunomodulatory effects, metabolites of IDO1 such as Kyn and quinolinic acid are also known to be neurotoxic and are observed to be elevated in several conditions of neurological dysfunction and depression. As such, IDO1 is a therapeutic target for inhibition in a broad array of indications, such as to promote tumor clearance, enable clearance of intractable viral or bacterial infections, decrease systemic immune dysfunction manifest as persistent inflammation during HIV infection or immunosuppression during sepsis, and prevent or reverse neurological conditions.

### IDO1 and Persistent Inflammation in HIV Infection:

Despite the success of antiretroviral therapy (ART) in suppressing HIV replication and decreasing the incidence of AIDS-related conditions, HIV-infected patients on ART have a higher incidence of non-AIDS morbidities and mortality than their uninfected peers. These non-AIDS conditions include cancer, cardiovascular disease, osteoporosis, liver disease, kidney disease, frailty, and neurocognitive dysfunction (Deeks 2011). Several studies indicate that non-AIDS morbidity/mortality is associated with persistent inflammation, which remains elevated in HIV-infected patients on ART as compared to peers (Deeks 2011). As such, it is hypothesized that persistent inflammation and immune dysfunction despite virologic suppression with ART is a cause of these non-AIDS-defining events (NADEs).

HIV infects and kills CD4⁺ T cells, with particular preference for cells like those CD4⁺ T cells that reside in the lymphoid tissues of the mucosal surfaces (Mattapallil, Douek et al. 2005). The loss of these cells combined with the inflammatory response to infection result in a perturbed relationship between the host and all pathogens, including HIV itself, but extending to pre-existing or acquired viral infections, fungal infections, and resident bacteria in the skin and mucosal surfaces. This dysfunctional host:pathogen relationship results in the over-reaction of the host to what would typically be minor problems as well as permitting the outgrowth of pathogens among the microbiota. The dysfunctional host:pathogen interaction therefore results in increased inflammation, which in turn leads to deeper dysfunction, driving a vicious cycle. As inflammation is thought to drive non-AIDS morbidity/mortality, the mechanisms governing the altered host:pathogen interaction are therapeutic targets.

IDO1 expression and activity are increased during untreated and treated HIV infection as well as in primate models of SIV infection (Boasso, Vaccari et al. 2007, Favre, Lederer et al. 2009, Byakwaga, Boum et al. 2014, Hunt, Sinclair et al. 2014, Tenorio, Zheng et al. 2014). IDO1 activity, as indicated by the ratio of plasma levels of enzyme substrate and product (Kyn/Tryp or K:T ratio), is associated with other markers of inflammation and is one of the strongest predictors of non-AIDS morbidity/mortality (Byakwaga, Bourn et al. 2014, Hunt, Sinclair et al. 2014, Tenorio, Zheng et al. 2014). In addition, features consistent with the expected impact of increased IDO1 activity on the immune system are major features of HIV and SIV induced immune dysfunction, such as decreased T cell proliferative response to antigen and imbalance of Treg:Th17 in systemic and intestinal compartments (Favre, Lederer et al. 2009, Favre, Mold et al. 2010). As such, we and others hypothesize that IDO1 plays a role in driving the vicious cycle of immune dysfunction and inflammation associated with non-AIDS morbidity/mortality. Thus, we propose that inhibiting IDO1 will reduce inflammation and decrease the risk of NADEs in ART-suppressed HIV-infected persons.

IDO1 and Persistent Inflammation beyond HIV

As described above, inflammation associated with treated chronic HIV infection is a likely driver of multiple end organ diseases [Deeks 2011]. However, these end organ diseases are not unique to HIV infection and are in fact the common diseases of aging that occur at earlier ages in the HIV-infected population. In the uninfected general population inflammation of unknown etiology is a major correlate of morbidity and mortality [Pinti, 2016 #88]. Indeed many of the markers of inflammation are shared, such as IL-6 and CRP. If, as hypothesized above, IDO1 contributes to persistent inflammation in the HIV-infected population by inducing immune dysfunction in the GI tract or systemic tissues, then IDO1 may also contribute to inflammation and therefore end organ diseases in the broader population. These inflammation associated end organ diseases are exemplified by cardiovascular diseases, metabolic syndrome, liver disease (NAFLD, NASH), kidney disease, osteoporosis, and neurocognitive impairment. Indeed, the IDO1 pathway has links in the literature to liver disease (Vivoli abstracts at Italian Assoc. for the Study of the Liver Conference 2015], diabetes [Baban, 2010 #89], chronic kidney disease [Schefold, 2009 #90], cardiovascular disease [Mangge, 2014 #92;Mangge, 2014 #91], as well as general aging and all cause mortality [Pertovaara, 2006 #93]. As such, inhibition of IDO1 may have application in decreasing inflammation in the general population to decrease the incidence of specific end organ diseases associated with inflammation and aging.

### IDO1 and Oncology

IDO expression can be detected in a number of human cancers (for example; melanoma, pancreatic, ovarian, AML, CRC, prostate and endometrial) and correlates with poor prognosis (Munn 2011). Multiple immunosuppressive roles have been ascribed to the action of IDO, including the induction of Treg differentiation and hyper-activation, suppression of Teff immune response, and decreased DC function, all of which impair immune recognition and promote tumor growth (Munn 2011). IDO expression in human brain tumors is correlated with reduced survival. Orthotropic and transgenic glioma mouse models demonstrate a correlation between reduced IDO expression and reduced Treg infiltration and an increased long term survival (Wainwright, Balyasnikova et al. 2012). In human melanoma a high proportion of tumors (33 of 36 cases) displayed elevated IDO suggesting an important role in establishing an immunosuppressive tumor microenvironment (TME) characterized by the expansion, activation and recruitment of MDSCs in a Treg-dependent manner (Holmgaard, Zamarin et al. 2015). Additionally, host IDO expressing immune cells have been identified in the draining lymph nodes and in the tumors themselves (Mellor and Munn 2004). Hence, both tumor and host-derived IDO are believed to contribute to the immune suppressed state of the TME.

The inhibition of IDO was one of the first small molecule drug strategies proposed for re-establishment of an immunogenic response to cancer (Mellor and Munn 2004). The d-enantiomer of 1-methyl tryptophan (D-1 MTor indoximod) was the first IDO inhibitor to enter clinical trials. While this compound clearly does inhibit the activity of IDO, it is a very weak inhibitor of the isolated enzyme and the in vivo mechanism(s) of action for this compound are still being elucidated. Investigators at Incyte optimized a hit compound obtained from a screening process into a potent and selective inhibitor with sufficient oral exposure to demonstrate a delay in tumor growth in a mouse melanoma model (Yue, Douty et al. 2009). Further development of this series led to INCB204360 which is a highly selective for inhibition of IDO-1 over IDO-2 and TDO in cell lines transiently transfected with either human or mouse enzymes (Liu, Shin et al. 2010). Similar potency was seen for cell lines and primary human tumors which endogenously express IDO1 (OC50s˜3-20 nM). When tested in co-culture of DCs and naïve CD4⁺CD25⁻ T cells, INCB204360 blocked the conversion of these T cells into CD4⁺FoxP3⁺ Tregs. Finally, when tested in a syngeneic model (PANO2 pancreatic cells) in immunocompetent mice, orally dosed INCB204360 provided a significant dose-dependent inhibition of tumor growth, but was without effect against the same tumor implanted in immune-deficient mice. Additional studies by the same investigators have shown a correlation of the inhibition of IDO1 with the suppression of systemic kynurenine levels and inhibition of tumor growth in an additional syngeneic tumor model in immunocompetent mice. Based upon these preclinical studies, INCB24360 entered clinical trials for the treatment of metastatic melanoma (Beatty, O'Dwyer et al. 2013).

In light of the importance of the catabolism of tryptophan in the maintenance of immune suppression, it is not surprising that overexpression of a second tryptophan metabolizing enzyme, TDO2, by multiple solid tumors (for example, bladder and liver carcinomas, melanomas) has also been detected. A survey of 104 human cell lines revealed 20/104 with TDO expression, 17/104 with IDO1 and 16/104 expressing both (Pilotte, Larrieu et al. 2012). Similar to the inhibition of IDO1, the selective inhibition of TDO2 is effective in reversing immune resistance in tumors overexpressing TDO2 (Pilotte, Larrieu et al. 2012). These results support TDO2 inhibition and/or dual TDO2/IDO1 inhibition as a viable therapeutic strategy to improve immune function.

Multiple pre-clinical studies have demonstrated significant, even synergistic, value in combining IDO-1 inhibitors in combination with T cell checkpoint modulating mAbs to CTLA-4, PD-1, and GITR. In each case, both efficacy and related PD aspects of improved immune activity/function were observed in these studies across a variety of murine models (Balachandran, Cavnar et al. 2011, Holmgaard, Zamarin et al. 2013, M. Mautino 2014, Wainwright, Chang et al. 2014). The Incyte IDO1 inhibitor (INCB204360, epacadostat) has been clinically tested in combination with a CTLA4 blocker (ipilimumab), but it is unclear that an effective dose was achieved due to dose-limited adverse events seen with the combination. In contrast recently released data for an on-going trial combining epacadostat with Merck's PD-1 mAb (pembrolizumab) demonstrated improved tolerability of the combination allowing for higher doses of the IDO1 inhibitor. There have been several clinical responses across various tumor types which is encouraging. However, it is not yet known if this combination is an improvement over the single agent activity of pembrolizumab (Gangadhar, Hamid et al. 2015). Similarly, Roche/Genentech are advancing NGL919/GDC-0919 in combination with both mAbs for PD-L1 (MPDL3280A, Atezo) and OX-40 following the recent completion of a phase 1a safety and PK/PD study in patients with advanced tumors.

IDO1 and chronic infections

IDO1 activity generates kynurenine pathway metabolites such as Kyn and 3-HAA that impair at least T cell, NK cell, and macrophage activity (Munn, Shafizadeh et al. 1999, Frumento, Rotondo et al. 2002) (Sekkai, Guittet et al. 1997, Favre, Mold et al. 2010). Kyn levels or the Kyn/Tryp ratio are elevated in the setting of chronic HIV infection (Byakwaga, Bourn et al. 2014, Hunt, Sinclair et al. 2014, Tenorio, Zheng et al. 2014), HBV infection (Chen, Li et al. 2009), HCV infection (Larrea, Riezu-Boj et al. 2007, Asghar, Ashiq et al. 2015), and TB infection(Suzuki, Suda et al. 2012) and are associated with antigen-specific T cell dysfunction (Boasso, Herbeuval et al. 2007, Boasso, Hardy et al. 2008, Loughman and Hunstad 2012, Ito, Ando et al. 2014, Lepiller, Soulier et al. 2015). As such, it is thought that in these cases of chronic infection, IDO1-mediated inhibition of the pathogen-specific T cell response plays a role in the persistence of infection, and that inhibition of IDO1 may have a benefit in promoting clearance and resolution of infection.

### IDO1 and Sepsis

IDO1 expression and activity are observed to be elevated during sepsis and the degree of Kyn or Kyn/Tryp elevation corresponded to increased disease severity, including mortality (Tattevin, Monnier et al. 2010, Darcy, Davis et al. 2011). In animal models, blockade of IDO1 or IDO1 genetic knockouts protected mice from lethal doses of LPS or from mortality in the cecal ligation/puncture model (Jung, Lee et al. 2009, Hoshi, Osawa et al. 2014). Sepsis is characterized by an immunosuppressive phase in severe cases (Hotchkiss, Monneret et al. 2013), potentially indicating a role for IDO1 as a mediator of immune dysfunction, and indicating that pharmacologic inhibition of IDO1 may provide a clinical benefit in sepsis.

### IDO1 and Neurological Disorders

In addition to immunologic settings, IDO1 activity is also linked to disease in neurological settings (reviewed in Lovelace Neuropharmacology 2016(Lovelace, Varney et al. 2016)). Kynurenine pathway metabolites such as 3-hydroxykynurenine and quinolinic acid are neurotoxic, but are balanced by alternative metabolites kynurenic acid or picolinic acid, which are neuroprotective. Neurodegenerative and psychiatric disorders in which kynurenine pathway metabolites have been demonstrated to be associated with disease include multiple sclerosis, motor neuron disorders such as amyotrophic lateral sclerosis, Huntington's disease, Parkinson's disease, Alzheimer's disease, major depressive disorder, schizophrenia, anorexia (Lovelace, Varney et al. 2016). Animal models of neurological disease have shown some impact of weak IDO1 inhibitors such as 1-methyltryptophan on disease, indicating that IDO1 inhibition may provide clinical benefit in prevention or treatment of neurological and psychiatric disorders.

It would therefore be an advance in the art to discover IDO inhibitors that effective the balance of the aforementioned properties as a disease modifying therapy in chronic HIV infections to decrease the incidence of non-AIDS morbidity/mortality; and/or a disease modifying therapy to prevent mortality in sepsis; and/or an immunotherapy to enhance the immune response to HIV, HBV, HCV and other chronic viral infections, chronic bacterial infections, chronic fungal infections, and to tumors; and/or for the treatment of depression or other neurological/ neuropsychiatric disorders.
Asghar, K., M. T. Ashiq, B. Zulfiqar, A. Mahroo, K. Nasir and S. Murad (2015). “Indoleamine 2,3-dioxygenase expression and activity in patients with hepatitis C virus-induced liver cirrhosis.” Exp Ther Med 9(3): 901-904.
Balachandran, V. P., M. J. Cavnar, S. Zeng, Z. M. Bamboat, L. M. Ocuin, H. Obaid, E. C. Sorenson, R. Popow, C. Ariyan, F. Rossi, P. Besmer, T. Guo, C. R. Antonescu, T. Taguchi, J. Yuan, J. D. Wolchok, J. P. Allison and R. P. Dematteo (2011). “Imatinib potentiates antitumor T cell responses in gastrointestinal stromal tumor through the inhibition of Ido.” Nature Medicine 17(9): 1094-1100.
Beatty, G. L., P. J. O′Dwyer, J. Clark, J. G. Shi, R. C. Newton, R. Schaub, J. Maleski, L. Leopold and T. Gajewski (2013). “Phase I study of the safety, pharmacokinetics (PK), and pharmacodynamics (PD) of the oral inhibitor of indoleamine 2,3-dioxygenase (IDO1) INCB024360 in patients (pts) with advanced malignancies.” ASCO Meeting Abstracts 31(15_suppl): 3025.
Boasso, A., A. W. Hardy, S. A. Anderson, M. J. Dolan and G. M. Shearer (2008). “HIV-induced type I interferon and tryptophan catabolism drive T cell dysfunction despite phenotypic activation.” PLoS One 3(8): e2961.
Boasso, A., J. P. Herbeuval, A. W. Hardy, S. A. Anderson, M. J. Dolan, D. Fuchs and G. M. Shearer (2007). “HIV inhibits CD4⁺ T-cell proliferation by inducing indoleamine 2,3-dioxygenase in plasmacytoid dendritic cells.” Blood 109(8): 3351-3359.
Boasso, A. and G. M. Shearer (2008). “Chronic innate immune activation as a cause of HIV-1 immunopathogenesis.” Clin Immunol 126(3): 235-242.
Boasso, A., M. Vaccari, A. Hryniewicz, D. Fuchs, J. Nacsa, V. Cecchinato, J. Andersson, G. Franchini, G. M. Shearer and C. Chougnet (2007). “Regulatory T-cell markers, indoleamine 2,3-dioxygenase, and virus levels in spleen and gut during progressive simian immunodeficiency virus infection.” J Virol 81(21): 11593-11603.
Byakwaga, H., Y. Boum, 2nd, Y. Huang, C. Muzoora, A. Kembabazi, S. D. Weiser, J. Bennett, H. Cao, J. E. Haberer, S. G. Deeks, D. R. Bangsberg, J. M. McCune, J. N. Martin and P. W. Hunt (2014). “The kynurenine pathway of tryptophan catabolism, CD4⁺ T-cell recovery, and mortality among HIV-infected Ugandans initiating antiretroviral therapy.” J Infect Dis 210(3): 383-391.
Chen, Y. B., S. D. Li, Y. P. He, X. J. Shi, Y. Chen and J. P. Gong (2009). “Immunosuppressive effect of IDO on T cells in patients with chronic hepatitis B*.” Hepatol Res 39(5): 463-468.
Darcy, C. J., J. S. Davis, T. Woodberry, Y. R. McNeil, D. P. Stephens, T. W. Yeo and N. M. Anstey (2011). “An observational cohort study of the kynurenine to tryptophan ratio in sepsis: association with impaired immune and microvascular function.” PLoS One 6(6): e21185.
Deeks, S. G. (2011). “HIV infection, inflammation, immunosenescence, and aging.” Annu Rev Med 62: 141-155.
Favre, D., S. Lederer, B. Kanwar, Z. M. Ma, S. Proll, Z. Kasakow, J. Mold, L. Swainson, J. D. Barbour, C. R. Baskin, R. Palermo, I. Pandrea, C. J. Miller, M. G. Katze and J. M. McCune (2009). “Critical loss of the balance between Th17 and T regulatory cell populations in pathogenic SIV infection.” PLoS Pathog 5(2): e1000295.
Favre, D., J. Mold, P. W. Hunt, B. Kanwar, P. Loke, L. Seu, J. D. Barbour, M. M. Lowe, A. Jayawardene, F. Aweeka, Y. Huang, D. C. Douek, J. M. Brenchley, J. N. Martin, F. M. Hecht, S. G. Deeks and J. M. McCune (2010). “Tryptophan catabolism by indoleamine 2,3-dioxygenase 1 alters the balance of TH17 to regulatory T cells in HIV disease.” Sci Transl Med 2(32): 32ra36.
Frumento, G., R. Rotondo, M. Tonetti, G. Damonte, U. Benatti and G. B. Ferrara (2002). “Tryptophan-derived catabolites are responsible for inhibition of T and natural killer cell proliferation induced by indoleamine 2,3-dioxygenase.” J Exp Med 196(4): 459-468.
Gangadhar, T., 0. Hamid, D. Smith, T. Bauer, J. Wasser, J. Luke, A. Balmanoukian, D. Kaufman, Y. Zhao, J. Maleski, L. Leopold and T. Gajewski (2015). “Preliminary results from a Phase I/II study of epacadostat (incb024360) in combination with pembrolizumab in patients with selected advanced cancers.” Journal for ImmunoTherapy of Cancer 3(Suppl 2): O7.
Holmgaard, R. B., D. Zamarin, Y. Li, B. Gasmi, D. H. Munn, J. P. Allison, T. Merghoub and J. D. Wolchok (2015). “Tumor-Expressed IDO Recruits and Activates MDSCs in a Treg-Dependent Manner.” Cell Reports 13(2): 412-424.
Holmgaard, R. B., D. Zamarin, D. H. Munn, J. D. Wolchok and J. P. Allison (2013). “Indoleamine 2,3-dioxygenase is a critical resistance mechanism in antitumor T cell immunotherapy targeting CTLA-4.” Journal of Experimental Medicine 210(7): 1389-1402.
Hoshi, M., Y. Osawa, H. Ito, H. Ohtaki, T. Ando, M. Takamatsu, A. Hara, K. Saito and M. Seishima (2014). “Blockade of indoleamine 2,3-dioxygenase reduces mortality from peritonitis and sepsis in mice by regulating functions of CD11b+peritoneal cells.” Infect Immun 82(11): 4487-4495.
Hotchkiss, R. S., G. Monneret and D. Payen (2013). “Sepsis-induced immunosuppression: from cellular dysfunctions to immunotherapy.” Nat Rev Immunol 13(12): 862-874.
Hunt, P. W., E. Sinclair, B. Rodriguez, C. Shive, B. Clagett, N. Funderburg, J. Robinson, Y. Huang, L. Epling, J. N. Martin, S. G. Deeks, C. L. Meinert, M. L. Van Natta, D. A. Jabs and M. M. Lederman (2014). “Gut epithelial barrier dysfunction and innate immune activation predict mortality in treated HIV infection.” J Infect Dis 210(8): 1228-1238. Ito, H., T. Ando, K. Ando, T. Ishikawa, K. Saito, H. Moriwaki and M. Seishima (2014). “Induction of hepatitis B virus surface antigen-specific cytotoxic T lymphocytes can be up-regulated by the inhibition of indoleamine 2, 3-dioxygenase activity.” Immunology 142(4): 614-623.
Jung, I. D., M. G. Lee, J. H. Chang, J. S. Lee, Y. I. Jeong, C. M. Lee, W. S. Park, J. Han, S. K. Seo, S. Y. Lee and Y. M. Park (2009). “Blockade of indoleamine 2,3-dioxygenase protects mice against lipopolysaccharide-induced endotoxin shock.” J Immunol 182(5): 3146-3154.
Larrea, E., J. I. Riezu-Boj, L. Gil-Guerrero, N. Casares, R. Aldabe, P. Sarobe, M. P. Civeira, J. L. Heeney, C. Rollier, B. Verstrepen, T. Wakita, F. Borras-Cuesta, J. J. Lasarte and J. Prieto (2007). “Upregulation of indoleamine 2,3-dioxygenase in hepatitis C virus infection.” J Virol 81(7): 3662-3666.
Lepiller, Q., E. Soulier, Q. Li, M. Lambotin, J. Berths, D. Fuchs, F. Stoll-Keller, T. J. Liang and H. Barth (2015). “Antiviral and Immunoregulatory Effects of Indoleamine-2,3-Dioxygenase in Hepatitis C Virus Infection.” J Innate Immun 7(5): 530-544.
Li, L., L. Huang, H. P. Lemos, M. Mautino and A. L. Mellor (2012). “Altered tryptophan metabolism as a paradigm for good and bad aspects of immune privilege in chronic inflammatory diseases.” Front Immunol 3: 109.
Liu, X., N. Shin, H. K. Koblish, G. Yang, Q. Wang, K. Wang, L. Leffet, M. J. Hansbury, B. Thomas, M. Rupar, P. Waeltz, K. J. Bowman, P. Polam, R. B. Sparks, E. W. Yue, Y. Li, R. Wynn, J. S. Fridman, T. C. Burn, A. P. Combs, R. C. Newton and P. A. Scherle (2010). “Selective inhibition of IDO1 effectively regulates mediators of antitumor immunity.” Blood 115(17): 3520-3530.
Loughman, J. A. and D. A. Hunstad (2012). “Induction of indoleamine 2,3-dioxygenase by uropathogenic bacteria attenuates innate responses to epithelial infection.” J Infect Dis 205(12): 1830-1839.
Lovelace, M. D., B. Varney, G. Sundaram, M. J. Lennon, C. K. Lim, K. Jacobs, G. J. Guillemin and B. J. Brew (2016). “Recent evidence for an expanded role of the kynurenine pathway of tryptophan metabolism in neurological diseases.” Neuropharmacology.
M. Mautino, C. J. L., N. Vahanian, J. Adams, C. Van Allen, M. D. Sharma, T. S. Johnson and D. H. Munn (2014). “Synergistic antitumor effects of combinatorial immune checkpoint inhibition with anti-PD-1/PD-L antibodies and the IDO pathway inhibitors NLG919 and indoximod in the context of active immunotherapy.” April 2014 AACR Meeting Poster # 5023.
Mattapallil, J. J., D. C. Douek, B. Hill, Y. Nishimura, M. Martin and M. Roederer (2005). “Massive infection and loss of memory CD4⁺ T cells in multiple tissues during acute SIV infection.” Nature 434(7037): 1093-1097.
Mellor, A. L. and D. H. Munn (2004). “IDO expression by dendritic cells: Tolerance and tryptophan catabolism.” Nature Reviews Immunology 4(10): 762-774.
Munn, D. H. (2011). “Indoleamine 2,3-dioxygenase, Tregs and cancer.” Current Medicinal Chemistry 18(15): 2240-2246.
Munn, D. H., E. Shafizadeh, J. T. Attwood, I. Bondarev, A. Pashine and A. L. Mellor (1999). “Inhibition of T cell proliferation by macrophage tryptophan catabolism.” J Exp Med 189(9): 1363-1372.
Pilotte, L., P. Larrieu, V. Stroobant, D. Colau, E. Dolušić, R. Frédérick, E. De Plaen, C. Uyttenhove, J. Wouters, B. Masereel and B. J. Van Den Eynde (2012). “Reversal of tumoral immune resistance by inhibition of tryptophan 2,3-dioxygenase.” Proceedings of the National Academy of Sciences of the United States of America 109(7): 2497-2502.
Sekkai, D., 0. Guittet, G. Lemaire, J. P. Tenu and M. Lepoivre (1997). “Inhibition of nitric oxide synthase expression and activity in macrophages by 3-hydroxyanthranilic acid, a tryptophan metabolite.” Arch Biochem Biophys 340(1): 117-123.
Suzuki, Y., T. Suda, K. Asada, S. Miwa, M. Suzuki, M. Fujie, K. Furuhashi, Y. Nakamura, N. Inui, T. Shirai, H. Hayakawa, H. Nakamura and K. Chida (2012). “Serum indoleamine 2,3-dioxygenase activity predicts prognosis of pulmonary tuberculosis.” Clin Vaccine Immunol 19(3): 436-442.
Tattevin, P., D. Monnier, 0. Tribut, J. Dulong, N. Bescher, F. Mourcin, F. Uhel, Y. Le Tulzo and K. Tarte (2010). “Enhanced indoleamine 2,3-dioxygenase activity in patients with severe sepsis and septic shock.” J Infect Dis 201(6): 956-966.
Tenorio, A. R., Y. Zheng, R. J. Bosch, S. Krishnan, B. Rodriguez, P. W. Hunt, J. Plants, A. Seth, C. C. Wilson, S. G. Deeks, M. M. Lederman and A. L. Landay (2014). “Soluble markers of inflammation and coagulation but not T-cell activation predict non-AIDS-defining morbid events during suppressive antiretroviral treatment.” J Infect Dis 210(8): 1248-1259.
Wainwright, D. A., I. V. Balyasnikova, A. L. Chang, A. U. Ahmed, K.-S. Moon, B. Auffinger, A. L. Tobias, Y. Han and M. S. Lesniak (2012). “IDO Expression in Brain Tumors Increases the Recruitment of Regulatory T Cells and Negatively Impacts Survival.” Clinical Cancer Research 18(22): 6110-6121.
Wainwright, D. A., A. L. Chang, M. Dey, I. V. Balyasnikova, C. K. Kim, A. Tobias, Y. Cheng, J. W. Kim, J. Qiao, L. Zhang, Y. Han and M. S. Lesniak (2014). “Durable therapeutic efficacy utilizing combinatorial blockade against IDO, CTLA-4, and PD-L1 in mice with brain tumors.” Clinical Cancer Research 20(20): 5290-5301.
Yue, E. W., B. Douty, B. Wayland, M. Bower, X. Liu, L. Leffet, Q. Wang, K. J. Bowman, M. J. Hansbury, C. Liu, M. Wei, Y. Li, R. Wynn, T. C. Burn, H. K. Koblish, J. S. Fridman, B. Metcalf, P. A. Scherle and A. P. Combs (2009). “Discovery of potent competitive inhibitors of indoleamine 2,3-dioxygenase with in vivo pharmacodynamic activity and efficacy in a mouse melanoma model.” Journal of Medicinal Chemistry 52(23): 7364-7367.

### SUMMARY OF THE INVENTION

Briefly, in one aspect, the present invention discloses compounds of Formula I

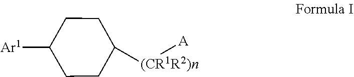

or a pharmaceutically acceptable salt thereof wherein:

Ar¹ is C₅₋₁₂aryl, or 5-12 membered heteroaryl, wherein aryl and heteroaryl include bicycles and heteroaryl contains 1-3 hetero atoms selected from O, S, and N, and wherein Ar¹ may optionally be substituted with 1-2 substituents independently selected from halogen, OH, C₁₋₃alkyl, OC₁₋₃alkyl, OC₁₋₃alkyl, C₁₋₃fluoroalkyl, CN, and NH₂;

R¹ and R² are independently H or C₁₋₄alkyl;

n is 1 or 0;

A is —C(O)NR³R⁴—, —NR⁴C(O)R³—, —NR⁴C(O)C(R⁷)(R⁸)R³—, or Ar²-R⁵, wherein Ar² is C₅₋₁₂aryl, or 5-12 membered heteroaryl, wherein aryl and heteroaryl include bicycles and heteroaryl contains 1-3 hetero atoms selected from O, S, and N, and wherein Ar² may optionally be substituted with a substituent selected from halogen, OH, C₁₋₃alkyl, OC₁₋₃alkyl, C₁₋₃fluoroalkyl, CN, and NH₂;

R⁴, R⁷, and R⁵ are independently H or C₁₋₆alkyl;

R⁵ is H, C₁₋₆alkyl, C₅₋₇aryl, optionally substituted with a substituent selected from the group consisting of halogen, C₁₋₄alkyl, hydroxyl, —C(O)CH₃, C(O)OCH₃, and C(O)NH₂.

R³ is C₁₋₁₀alkyl, C₃₋₈cycloalkyl, or C₅₋₇aryl wherein R³ is optionally substituted with a substituent selected from the group consisting of halogen, C₁₋₄alkyl, hydroxyl, —C(O)CH₃, C(O)OCH₃, and C(O)NH₂.

In another aspect, the present invention discloses a method for treating diseases or conditions that would benefit from inhibition of IDO.

In another aspect, the present invention discloses pharmaceutical compositions comprising a compound of Formula I or a pharmaceutically acceptable salt thereof.

In another aspect, the present invention provides a compound of Formula I or a pharmaceutically acceptable salt thereof for use in therapy.

In another aspect, the present invention provides a compound of Formula I or a pharmaceutically acceptable salt thereof for use in treating diseases or condition that would benefit from inhibition of IDO.

In another aspect, the present invention provides use of a compound of Formula I or a pharmaceutically acceptable salt thereof in the manufacture of a medicament for use in treating diseases or conditions that would benefit from inhibition of IDO.

In another aspect, the present invention discloses a method for treating a viral infection in a patient mediated at least in part by a virus in the retrovirus family of viruses, comprising administering to said patient a composition comprising a compound of Formula I, or a pharmaceutically acceptable salt thereof. In some embodiments, the viral infection is mediated by the HIV virus.

In another aspect, a particular embodiment of the present invention provides a method of treating a subject infected with HIV comprising administering to the subject a therapeutically effective amount of a compound of Formula I, or a pharmaceutically acceptable salt thereof.

In yet another aspect, a particular embodiment of the present invention provides a method of inhibiting progression of HIV infection in a subject at risk for infection with HIV comprising administering to the subject a therapeutically effective amount of a compound of Formula I, or a pharmaceutically acceptable salt thereof. Those and other embodiments are further described in the text that follows.

### DETAILED DESCRIPTION OF REPRESENTATIVE EMBODIMENTS

Preferably Ar¹ is quinoline, isoquinoline, quinazoline, isoquinolone, quinazolone, naphthyridine, naphthalene, or indole, and may optionally be substituted with a substituent selected from halogen, OH, C₁₋₃alkyl, OC₁₋₃alkyl, C₁₋₃fluoroalkyl, CN, and NH₂. More preferably Ar¹ is quinoline optionally substituted with a halogen. Most preferably Ar¹ is unsubstituted quinoline.

Preferably R¹ and R² are independently H or methyl.

Preferably Ar² is unsubstituted benzimidazole, 7-chloro-benzimidazole, oxazole, imidazole, 1,2,4-triazole, benzoxazolone, or benzoimidazolone. More preferably Ar² is unsubstituted benzimidazole or imidazole.

Preferably R⁵ is H, C₁₋₆alkyl, or phenyl optionally substituted with a halogen.

Preferably R³ is C₁₋₁₀alkyl, C₅₋₇cycloalkyl, or phenyl wherein R³ is optionally substituted with a substituent selected from the group consisting of halogen, C₁₋₃alkyl, hydroxyl, and C(O)NH₂.

Preferred pharmaceutical compositions include unit dosage forms. Preferred unit dosage forms include tablets.

It is expected that the compounds and composition of this invention will be useful for prevention and/or treatment of HIV; including the prevention of the progression of AIDS and general immunosuppression. It is expected that in many cases such prevention and/or treatment will involve treating with the compounds of this invention in combination with at least one other drug thought to be useful for such prevention and/or treatment. For example, the IDO inhibitors of this invention may be used in combination with other immune therapies such as immune checkpoints (PD1, CTLA4, ICOS, etc.) and possibly in combination with growth factors or cytokine therapies (IL21, IL-7, etc.).

In is common practice in treatment of HIV to employ more than one effective agent. Therefore, in accordance with another embodiment of the present invention, there is provided a method for preventing or treating a viral infection in a mammal mediated at least in part by a virus in the retrovirus family of viruses which method comprises administering to a mammal, that has been diagnosed with said viral infection or is at risk of developing said viral infection, a compound as defined in Formula I, wherein said virus is an HIV virus and further comprising administration of a therapeutically effective amount of one or more agents active against an HIV virus, wherein said agent active against the HIV virus is selected from the group consisting of Nucleotide reverse transcriptase inhibitors; Non-nucleotide reverse transcriptase inhibitors; Protease inhibitors; Entry, attachment and fusion inhibitors; Integrase inhibitors; Maturation inhibitors; CXCR4 inhibitors; and CCR5 inhibitors. Examples of such additional agents are Dolutegravir, Bictegravir, and Cabotegravir.

“Pharmaceutically acceptable salt” refers to pharmaceutically acceptable salts derived from a variety of organic and inorganic counter ions well known in the art and include, by way of example only, sodium, potassium, calcium, magnesium, ammonium, and tetraalkylammonium, and when the molecule contains a basic functionality, salts of organic or inorganic acids, such as hydrochloride, hydrobromide, tartrate, mesylate, acetate, maleate, and oxalate. Suitable salts include those described in P. Heinrich Stahl, Camille G. Wermuth (Eds.), Handbook of Pharmaceutical Salts Properties, Selection, and Use; 2002.

The present invention also includes pharmaceutically acceptable salts of the compounds described herein. As used herein, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present invention include the conventional non-toxic salts of the parent compound formed, for example, from non-toxic inorganic or organic acids. The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or ACN are preferred.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

In one embodiment, the pharmaceutical formulation containing a compound of Formula I or a salt thereof is a formulation adapted for oral or parenteral administration. In another embodiment, the formulation is a long-acting parenteral formulation. In a further embodiment, the formulation is a nano-particle formulation.

The present invention is directed to compounds, compositions and pharmaceutical compositions that have utility as novel treatments for immunosuppresion. While not wanting to be bound by any particular theory, it is thought that the present compounds are able to inhibit the enzyme that catalyzes the oxidative pyrrole ring cleavage reaction of I-Trp to N-formylkynurenine utilizing molecular oxygen or reactive oxygen species.

Therefore, in another embodiment of the present invention, there is provided a method for the prevention and/or treatment of HIV; including the prevention of the progression of AIDS and general immunosuppression.

### EXAMPLES

The following examples serve to more fully describe the manner of making and using the above-described invention. It is understood that these examples in no way serve to limit the true scope of the invention, but rather are presented for illustrative purposes. In the examples and the synthetic schemes below, the following abbreviations have the following meanings. If an abbreviation is not defined, it has its generally accepted meaning.
ACN=Acetonitrile
AIBN=azobisisobutyronitrile
aq.=Aqueous
μL or uL=Microliters
μM or uM=Micromolar
NMR=nuclear magnetic resonance
boc=tert-butoxycarbonyl
br=Broad
Cbz=Benzyloxycarbonyl
CDl=1,1′-carbonyldiimidazole
d=Doublet
δ=chemical shift
° C.=degrees celcius
DCM=dichloromethane
dd=doublet of doublets
DHP=dihydropyran
DIAD=diisopropyl azodicarboxylate
DIEA or DIPEA=N,N-diisopropylethylamine
DMAP=4-(dimethylamino)pyridine
DMEM=Dulbeco's Modified Eagle's Medium
EtOAc=ethyl acetate
h or hr=Hours
HATU=1-[Bis(dimethylamino)methylene]-1H-1,2,3-triazolo[4,5-b]pyridinium 3-oxid hexafluorophosphate
HCV=hepatitis C virus
HPLC=high performance liquid chromatography
Hz=Hertz
IU=International Units
IC50=inhibitory concentration at 50% inhibition
J=coupling constant (given in Hz unless otherwise indicated)
LCMS=liquid chromatography-mass spectrometry
m=Multiplet
M=Molar
M+H+=parent mass spectrum peak plus H+
MeOH=Methanol
mg=Milligram
min=Minutes
mL=Milliliter
mM=Millimolar
mmol=Millimole
MS=mass spectrum
MTBE=methyl tent-butyl ether
N=Normal
NFK=N- formylkynurenine
NBS=N-bromosuccinimide
nm=Nanomolar
PE=petroleum ether
ppm=parts per million
q.s.=sufficient amount
s=Singlet
RT=room temperature
Rf=retardation factor
sat.=Saturated
t=Triplet
TEA=triethylamine
TFA=trifluoroacetic acid
TFAA=trifluoroacetic anhydride
THF=tetrahydrofuran

### Equipment Description

¹H NMR spectra were recorded on a Bruker Ascend 400 spectrometer or a Varian 400 spectrometer. Chemical shifts are expressed in parts per million (ppm, δ units). Coupling constants are in units of hertz (Hz). Splitting patterns describe apparent multiplicities and are designated as s (singlet), d (doublet), t (triplet), q (quartet), quint (quintet), m (multiplet), br (broad).

The analytical low-resolution mass spectra (MS) were recorded on Waters ACQUITY UPLC with SQ Detectors using a Waters BEH C18, 2.1×50 mm, 1.7 μm using a gradient elution method.

Solvent A: 0.1% formic acid (FA) in water;

Solvent B: 0.1% FA in acetonitrile;

30% B for 0.5 min followed by 30-100% B over 2.5 min.

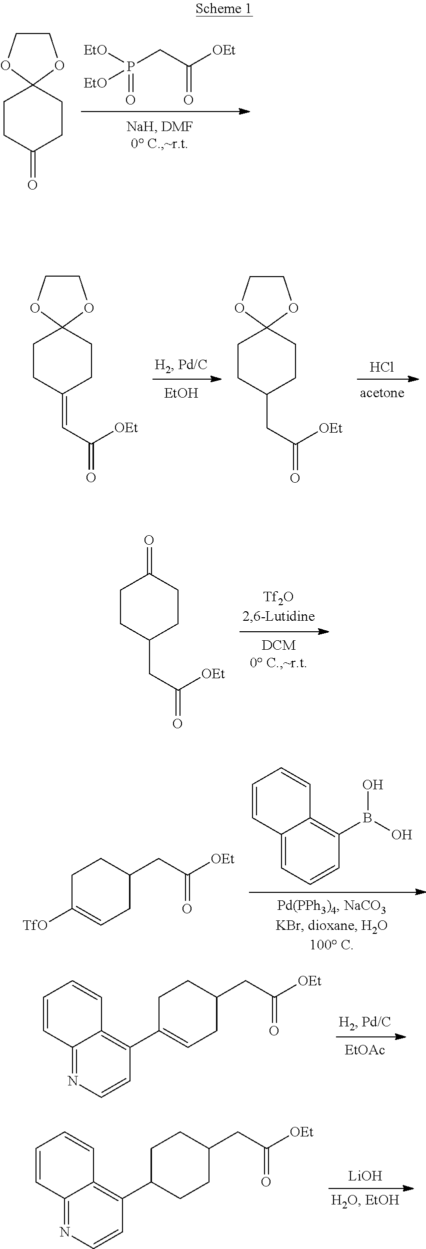

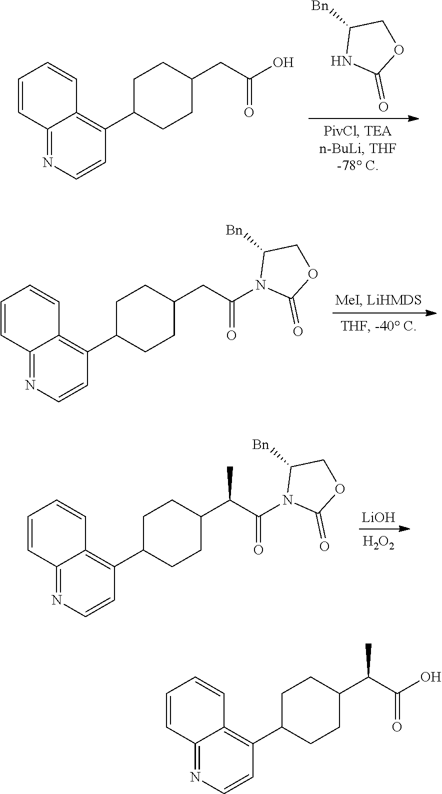

### Preparation of ethyl 2-(1,4-dioxaspiro[4.5]decan-8-ylidene)acetate

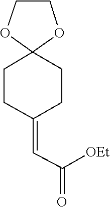 [SMILES: CCOC(=O)C=C1CCC2(CC1)OCCO2]

At 0° C., to a suspension of NaH (60% in oil) (6.92 g, 288 mmol) in anhydrous THF (650 mL) under nitrogen with vigorous stirring was added the triethyl phosphonoacetate (52.5 g, 288 mmol.) dropwise. After stirred at 0° C. for 30 min, 1,4-cyclohexanedione monoethylene ketal (41 g, 260 mmol) in THF (150 mL) was added dropwise. The resulting mixture was allowed to warm up to room temperature and stirred overnight. The reaction mixture was poured into saturated aq. NH₄Cl and extracted with EtOAc. The organics were washed sequentially with water and brine, and dried over Na₂SO₄. Filtration and concentration in vacuum gave a crude product, which was purified by flash chromatography (silica gel, 0-30% EtOAc in PE) to afford the title compound (56 g, 95% yield). (ESI) m/z calcd for C₁₂H₁₈O₄: 226.12. Found: 227.33 (M+1)⁺.

### Preparation of ethyl 2-(1,4-dioxaspiro[4.5]decan-8-yl)acetate

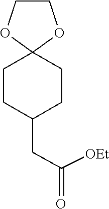 [SMILES: CCOC(=O)CC1CCC2(CC1)OCCO2]

A mixture of ethyl 2-(1,4-dioxaspiro[4.5]decan-8-ylidene)acetate (17.3 g, 76.4 mmol) and 10% Pd/C (5.19 g) in EtOH (500 mL) was stirred at room temperature under H₂ atmosphere (15 psi) overnight. The resulting mixture was filtered through a pad of Celite and the filtrate was concentrated under reduced pressure to afford the title compound (17.5 g, 100% yield), which was used in the following step without purification. (ESI) m/z calcd for C₁₂H₂₀O₄: 228.14. Found: 229.20 (M+1)⁺.

### Preparation of ethyl 2-(4-oxocyclohexyl)acetate

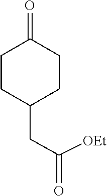 [SMILES: CCOC(=O)CC1CCC(=O)CC1]

To a solution of Methyl 2-(4-(1,3-dioxalane)cyclohexyl)acetate (17.5 g, 76.4 mmol) in acetone, was added 1 N HCl (160 mL, 160 mmol) dropwise. After the reaction mixture was stirred at room temperature overnight, water and EtOAc were added and the layers were separated. The organics were washed sequentially with water and brine, and dried over Na₂SO₄. Filtration and concentration in vacuum gave a crude product, which was purified by flash chromatography (silica gel, 0-30% EtOAc in PE) to afford the title compound (10 g, 72% yield). (ESI) m/z calcd for C₁₀H₁₆O₃: 184.11. Found: 185.34 (M+1)⁺.

### Preparation of ethyl 2-(4-(((trifluoromethyl)sulfonyl)oxy)cyclohex-3-en-1-yl)acetate

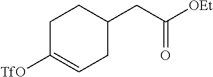 [SMILES: CCOC(=O)CC1CC=C(OS(=O)(=O)C(F)(F)F)CC1]

At OcC, to a solution of ethyl 2-(4-oxocyclohexyl)acetate (10 g, 54.3 mmol) and trifluoromethanesulfonic anhydride (18.4 g, 65.2 mmol) in dichloromethane, was added 2,6-dimethylpyridine (12.5 mL, 108.6 mmol) dropwise. The reaction mixture was stirred overnight at room temperature. Then this was partitioned between aq. NH₄Cl and EtOAc and the layers were separated. The organics were washed sequentially with water and brine, and dried over Na₂SO₄. Filtration and concentration in vacuum gave a crude product, which was purified by flash chromatography to afford the title compound (11.5 g, 67% yield). (ESI) m/z calcd for C₁₁H₁₅F₃O₅S: 316.06. Found: 317.19 (M+1)⁺.

### Preparation of ethyl 2-(4-(quinolin-4-yl)cyclohex-3-en-1-yl)acetate

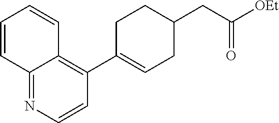 [SMILES: CCOC(=O)CC1CC=C(c2ccnc3ccccc23)CC1]

Ethyl 2-(4-(((trifluoromethyl)sulfonyl)oxy)cyclohex-3-en-1-yhacetate (10 g, 31.6 mmol), quinolin-4-ylboronic acid (8.2 g, 47.4 mmol), Pd(PPh₃)₄ (3.65 g, 3.16 mmol) and KBr (4.14 g, 34.8 mmol) were dissolved in dioxane (100 mL). After adding 2 M aqueous sodium carbonate solution (40 mL), the mixture was stirred under nitrogen atmosphere at 100° C. for 14 hours. After the reaction mixture was cooled to room temperature, this was partitioned between water and EtOAc and the layers were separated. The organics were washed sequentially with water and brine, and dried over Na₂SO₄. Filtration and concentration in vacuum gave a crude product, which was purified by flash chromatography to afford the title compound (5.4 g, 58% yield). (ESI) m/z calcd for C₁₉H₂₁NO₂: 295.16. Found: 296.58 (M+1)⁺.

### Preparation of ethyl 2-(4-(quinolin-4-yl)cyclohexyl)acetate

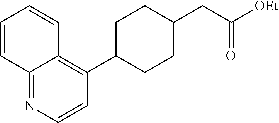 [SMILES: CCOC(=O)CC1CCC(c2ccnc3ccccc23)CC1]

A mixture of ethyl 2-(4-(quinolin-4-yl)cyclohex-3-en-1-yl)acetate (3 g, 10.2 mmol) and 10% Pd/C (1.5 g) in MeOH (300 mL) was stirred at room temperature under H₂ atmosphere (15 psi) overnight. The resulting mixture was filtered through a pad of Celite and the filtrate was concentrated under reduced pressure to give the crude product which was purified by flash chromatography (silica gel, 0-50% EtOAc in PE) to afford the title compound (1.8 g, 60% yield) as a brown oil. (ESI) m/z calcd for C₁₉H₂₃NO₂: 297.17. Found: 298.49 (M+1)⁺.

### Preparation of 2-(4-(quinolin-4-yl)cyclohexyl)acetic acid

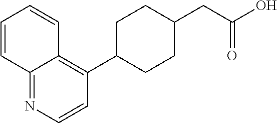 [SMILES: O=C(O)CC1CCC(c2ccnc3ccccc23)CC1]

To a solution of methyl 2-(3-((5-chloropyridin-2-yl)amino)-4-(isobutyl(tetrahydro-2H-pyran-4-yl)amino)phenyl)-2-methylpropanoate (1.8 g, 6.1 mmol) in EtOH (6 mL) was added 1N LiOH aq. (45 mL, 45 mmol). After stirred at 50° C. for 2h, the resulting mixture was neutralized with 1N HCl and extracted with EtOAc. The organic layer was washed with brine, dried over Na₂SO₄, filtered and concentrated to give the title compound (1.5 g, 95% yield) as a pale solid, which was used in the following step without further purification. LCMS (ESI) m/z calcd for C₁₇H₁₉NO₂: 269.14. Found: 270.51 (M+1)⁺.

### Preparation of (R)-4-benzyl-3-(2-(4-(quinolin-4-Acyclohexyl)acetyl)oxazolidin-2-one

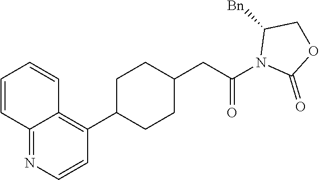 [SMILES: O=C(CC1CCC(c2ccnc3ccccc23)CC1)N1C(=O)OC[C@H]1Cc1ccccc1]

At −78° C., to a solution of 2-(4-(quinolin-4-yl)cyclohexyl)acetic acid (1.0 g, 3.7 mmol), TEA (1 mL, 7.4 mmol) in THF(15 mL) under nitrogen atmosphere (flask #1), was added pivaloyl chloride (551 mg, 4.6 mmol) drop wise over 15 min. The reaction mixture was then stirred at 0° C. for another 1 hour.

To a separate flask (flask #2), charged with (R)-4-benzyloxazolidin-2-one (850 mg, 4.8 mmol) and THF(20 mL) at −78° C., was added n-BuLi (2.0 mL, 4.8 mmol) drop wise. The reaction mixture was stirred at −78° C. for 15 min before being removed from the cold bath.

Flask #1 was cooled back to −78° C. and the solution in flask #2 was added to flask #1 vial cannula over 15 min. After complete addition, the cold bath was removed and the reaction mixture was stirred at room temperature for 3 hours. The reaction was quenched with sat. NH₄Cl solution and extracted with EtOAc. The organics were washed sequentially with water and brine, and dried over Na₂SO₄. Filtration and concentration in vacuum gave a crude product, which was purified by flash chromatography to afford the title compound (1.3 g, 67% yield). (ESI) m/z calcd for C₂₇H₂₈N₂O₃: 428.21. Found: 429.47 (M+1)⁺.

### Preparation of (R)-4-benzyl-3-((R)-2-(4-(quinolin-4-yl)cyclohexyl)propanoyl) oxazolidin-2-one

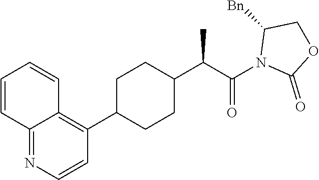 [SMILES: C[C@@H](C(=O)N1C(=O)OC[C@H]1Cc1ccccc1)C1CCC(c2ccnc3ccccc23)CC1]

At 0° C., to a solution of (R)-4-benzyl-3-(2-(4-(quinolin-4-yl)cyclohexyl)acetyl)oxazolidin-2-one (1.2 g, 2.8 mmol) in THF (15 mL) under nitrogen atmosphere, was added LiHMDS (5.6 mL, 5.6 mmol) drop wise over 15 min. The reaction mixture was stirred at 0° C. for 30 min, the reaction mixture was cooled to −40° C. before iodomethane (0.4 mL, 5.6 mmol) was added drop wise. After complete addition, the reaction mixture was stirred at this temperature for 20 hours. The reaction was quenched with sat. NH₄Cl solution and extracted with EtOAc. The organics were washed sequentially with water and brine, and dried over Na₂SO₄. Filtration and concentration in vacuum gave a crude product, which was purified by flash chromatography to afford the title compound (752 mg, 60% yield). (ESI) m/z calcd for C₂₈H₃₀N₂O₃: 442.23. Found: 443.52 (M+1)⁺.

### Preparation of (R)-2-(4-(quinolin-4-yl)cyclohexyl)propanoic acid

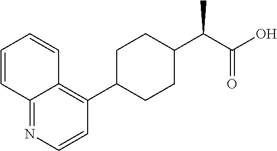 [SMILES: C[C@@H](C(=O)O)C1CCC(c2ccnc3ccccc23)CC1]

At 0° C., to a solution of methyl (R)-4-benzyl-3-((R)-2-(4-(quinolin-4-yl)cyclohexyl) propanoyl) oxazolidin-2-one (500 mg, 1.13 mmol) in THF (10 mL) was added 35% H₂O₂ (0.5 mL), followed by addition of 1M LiOH aq. (1.8 mL). After stirred at room temperature overnight, the resulting mixture was quenched with sat. Na₂SO₃ solution, neutralized with 1N HCl and extracted with EtOAc. The organic layer was washed with brine, dried over Na₂SO₄, filtered and concentrated to give the crude product, which was purified by flash chromatography to afford the title compound (270 mg, 84% yield). LCMS (ESI) m/z calcd for C₁₈H₂₁NO₂: 283.16. Found: 284.61 (M+1)⁺.

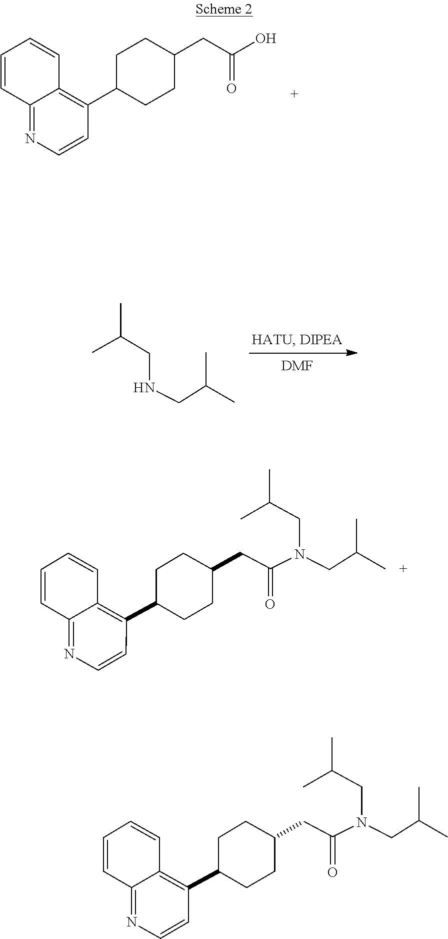

### Example 1 and Example 2

### Preparation of N,N-diisobutyl-2-(cis-4-(quinolin-4-yl)cyclohexyl)acetamide and N,N-diisobutyl-2-(trans-4-(quinolin-4-yl)cyclohexyl)acetamide

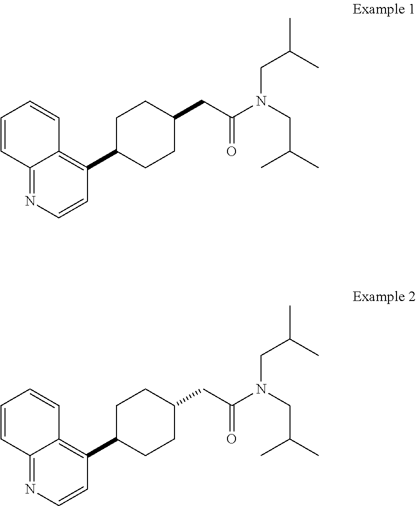 [SMILES: CC(C)CN(CC(C)C)C(=O)C[C@H]1CC[C@@H](c2ccnc3ccccc32)CC1 ; CC(C)CN(CC(C)C)C(=O)C[C@H]1CC[C@H](c2ccnc3ccccc32)CC1]

To a stirred solution of 2-(4-(quinolin-4-yl)cyclohexyl)acetic acid (300 mg, 1.11 mmol) and diisobutylamine (288 mg, 2.23 mmol) in DMF (6 mL) was added DIPEA (0.58 mL, 3.34 mmol) followed by HATU (847 mg, 2.23 mmol). After stirred at r.t. overnight, the reaction mixture was quenched with brine and the resulting mixture was extracted with DCM (x3). The combined organic layers were dried over Na₂SO₄. Solvent was removed under vacuum and the residue was purified by Prep. TLC (PE/THF=3/1) to afford the title compound. Example 1 cis-isomer (78 mg, 18% yield): ¹H NMR (400 MHz, CDCl₃) δ 8.79 (d, J=4.5 Hz, 1H), 8.04 (dd, J=20.8, 8.4 Hz, 2H), 7.64 (t, J=7.1 Hz, 1H), 7.50 (t, J=7.2 Hz, 1H), 7.27 (d, J=4.6 Hz, 1H), 3.39-3.31 (m, 1H), 3.15 (d, J=7.5 Hz, 2H), 3.07 (d, J=7.5 Hz, 2H), 2.43 (s, 3H), 1.98-1.88 (m, 2H), 1.86-1.67 (m, J=21.5, 15.5, 11.1 Hz, 8H), 0.88 (d, J=6.7 Hz, 6H), 0.80 (d, J=6.7 Hz, 6H). LCMS (ESI) m/z calcd for C₂₅H₃₆N₂O: 380.28. Found: 381.46 (M+1)⁺. Example 2 trans-isomer (14 mg, 3% yield): ¹H NMR (400 MHz, CDCl₃) δ 8.78 (d, J=4.6 Hz, 1H), 8.09-7.97 (m, 2H), 7.66-7.58 (m, 1H), 7.52-7.44 (m, 1H), 7.21 (d, J=4.6 Hz, 1H), 3.28-3.20 (m, 1H), 3.15 (d, J=7.5 Hz, 2H), 3.07 (d, J=7.6 Hz, 2H), 2.25 (d, J=6.6 Hz, 2H), 2.03 -1.92 (m, 5H), 1.59 (dd, J=23.5, 11.4 Hz, 4H), 1.26-1.21 (m, 2H), 0.88 (d, J=6.7 Hz, 6H), 0.82 (d, J=6.7 Hz, 6H). LCMS (ESI) m/z calcd for C₂₅H₃₆N₂O: 380.28. Found: 381.40 (M+1)⁺.

The following compounds in Table 1 were prepared similarly to the above procedures using appropriate amine.

TABLE 1

| Exam- | | Exact | M + 1 |
|---|---|---|---|
| ple | structure | mass | observed |
| 3 | 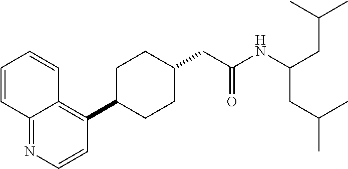 [SMILES: CC(C)CC(CC(C)C)NC(=O)C[C@H]1CC[C@H](c2ccnc3ccccc32)CC1] | 394.30 | 395.46 |
| 4 | 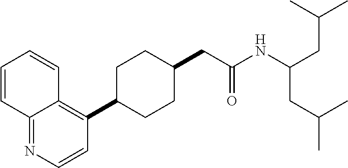 [SMILES: CC(C)CC(CC(C)C)NC(=O)C[C@H]1CC[C@@H](c2ccnc3ccccc32)CC1] | 394.30 | 395.37 |
| 5 | 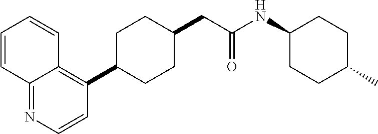 [SMILES: C[C@H]1CC[C@H](NC(=O)C[C@H]2CC[C@@H](c3ccnc4ccccc43)CC2)CC1] | 364.25 | 365.36 |
| 6 | 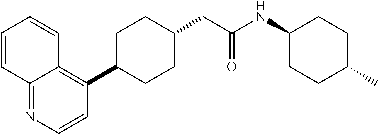 [SMILES: C[C@H]1CC[C@H](NC(=O)C[C@H]2CC[C@H](c3ccnc4ccccc43)CC2)CC1] | 364.25 | 365.36 |
| 7 | 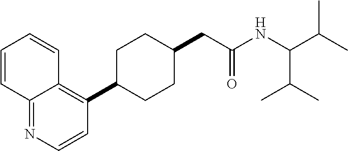 [SMILES: CC(C)C(NC(=O)C[C@H]1CC[C@@H](c2ccnc3ccccc32)CC1)C(C)C] | 366.27 | 367.39 |
| 8 | 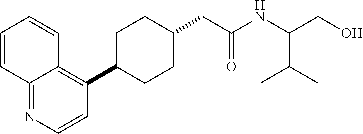 [SMILES: CC(C)C(CO)NC(=O)C[C@H]1CC[C@H](c2ccnc3ccccc32)CC1] | 354.23 | 355.32 |
| 9 | 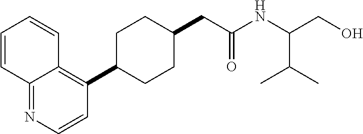 [SMILES: CC(C)C(CO)NC(=O)C[C@H]1CC[C@@H](c2ccnc3ccccc32)CC1] | 354.23 | 355.29 |
| 10 | 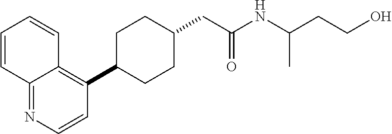 [SMILES: CC(CCO)NC(=O)C[C@H]1CC[C@H](c2ccnc3ccccc32)CC1] | 340.22 | 341.46 |
| 11 | 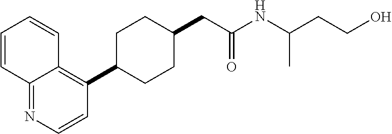 [SMILES: CC(CCO)NC(=O)C[C@H]1CC[C@@H](c2ccnc3ccccc32)CC1] | 340.22 | 341.47 |

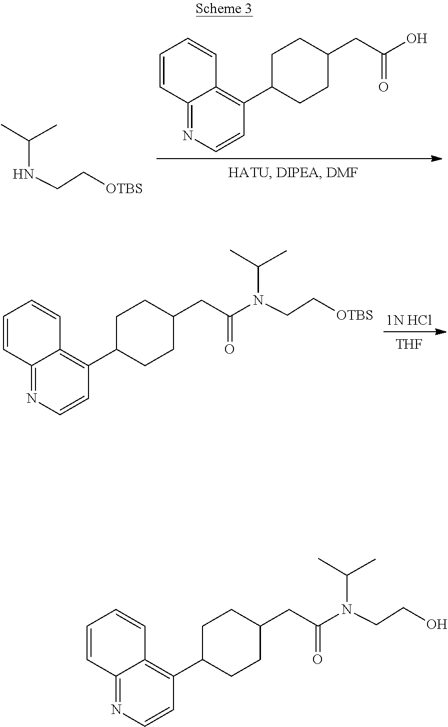

### Preparation of N-(2-((tert-butyldimethylsilyl)oxy)ethyl)-N-isopropyl-2-(4-(quinolin-4-yl)cyclohexyl)acetamide

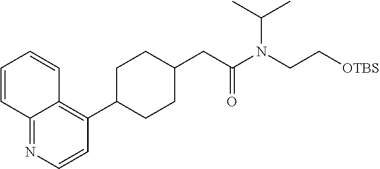 [SMILES: CC(C)N(CCO[Si](C)(C)C(C)(C)C)C(=O)CC1CCC(c2ccnc3ccccc23)CC1]

To a stirred solution of 2-(4-(quinolin-4-yl)cyclohexyl)acetic acid (180 mg, 0.67 mmol) and N-(2-((tert-butyldimethylsilyl)oxy)ethyl)propan-2-amine (145 mg, 0.67 mmol) in DMF (3 mL) was added DIPEA (0.36 mL, 2.01 mmol) followed by HATU (280 mg, 0.74 mmol). After stirred at r.t. overnight, the reaction mixture was quenched with brine and the resulting mixture was extracted with DCM (x3). The combined organic layers were dried over Na₂SO₄. Solvent was removed under vacuum and the residue was purified by column chromatography on silica gel to afford the title compound (200 mg, 67% yield). LCMS (ESI) m/z calcd for C₂₈H₄₄N₂O₂Si: 468.32. Found: 469.36 (M+1)⁺.

### Example 12

### Preparation of N-(2-hydroxyethyl)-N-isopropyl-2-(4-(quinolin-4-yl)cyclohexyl) acetamide

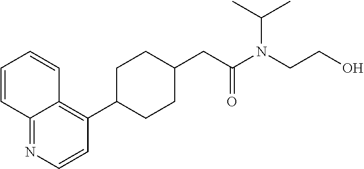 [SMILES: CC(C)N(CCO)C(=O)CC1CCC(c2ccnc3ccccc23)CC1]

To a stirred solution of N-(2-((tert-butyldimethylsilyl)oxy)ethyl)-N-isopropyl-2-(4-(quinolin-4-yl)cyclohexyl)acetamide (200 mg, 0.427 mmol) in THF (2 mL) was added 1N aq. HCl (2 mL). After stirred at room temperature for 1 hour, the reaction mixture was neutralized with 1N NaOH and extracted with EtOAc. The combined organic layers were dried over Na₂SO₄. Solvent was removed under vacuum and the residue was purified by column chromatography on silica gel to afford the title compound (92 mg, 61% yield) as a white solid. ¹H NMR (400 MHz, DMSO) δ 8.89-8.80 (m, 1H), 8.22 (d, J=8.4 Hz, 1H), 8.03 (d, J=8.3 Hz, 1H), 7.80-7.71 (m, 1H), 7.67-7.59 (m, 1H), 7.52-7.41 (m, 1H), 4.95-4.61 (m, 1H), 4.49-4.13 (m, 1H), 3.53-3.19 (m, 6H), 2.34-2.26 (m, 1H), 1.97-1.55 (m, 8H), 1.34-1.22 (m, 1H), 1.20-1.03 (m, 6H). LCMS (ESI) m/z calcd for C₂₂H₃₀N₂O₂: 354.23. Found: 355.32 (M+1)⁺.

### Example 13

### Preparation of N-(3-hydroxypropyl)-N-isopropyl-2-(4-(quinolin-4-yl)cyclohexyl) acetamide

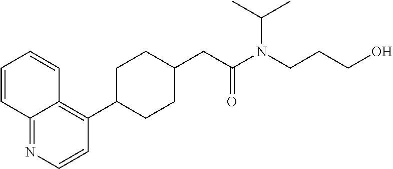 [SMILES: CC(C)N(CCCO)C(=O)CC1CCC(c2ccnc3ccccc23)CC1]

The title compound was prepared from 2-(4-(quinolin-4-yl)cyclohexyl)acetic acid and 3-(isopropylamino)propan-1-ol according to the procedure described for the synthesis of N-(2-hydroxyethyl)-N-isopropyl-2-(4-(quinolin-4-yl)cyclohexyl) acetamide (scheme 3). ¹H NMR (400 MHz, DMSO) δ 8.87-8.78 (m, 1H), 8.21 (d, J=8.4 Hz, 1H), 8.02 (d, J=8.3 Hz, 1H), 7.79-7.70 (m, 1H), 7.67-7.57 (m, 1H), 7.51-7.40 (m, 1H), 4.71-4.12 (m, 2H), 3.58-3.09 (m, 6H), 2.34-2.22 (m, 1H), 2.01-1.51 (m, 10H), 1.39-1.23 (m, 1H), 1.16 (d, J=6.6 Hz, 3H), 1.09 (d, J=6.8 Hz, 3H). LCMS (ESI) m/z calcd for C₂₃H₃₂N₂O₂: 368.25. Found: 369.53 (M+1)⁺.

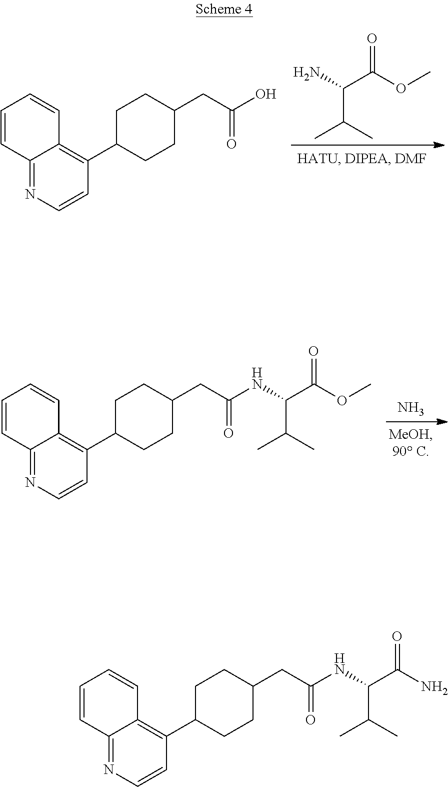

### Preparation of methyl (2-(4-(quinolin-4-yl)cyclohexyl)acetyl)-L-valinate

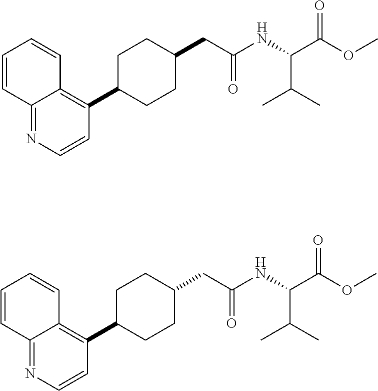 [SMILES: COC(=O)[C@@H](NC(=O)C[C@H]1CC[C@@H](c2ccnc3ccccc32)CC1)C(C)C ; COC(=O)[C@@H](NC(=O)C[C@H]1CC[C@H](c2ccnc3ccccc32)CC1)C(C)C]

To a stirred solution of 2-(4-(quinolin-4-yl)cyclohexyl)acetic acid (300 mg, 1.11 mmol) and methyl L-valinate (175 mg, 1.34 mmol) in DMF (3 mL) was added DIPEA (0.60 mL, 3.33 mmol) followed by HATU (464 mg, 1.22 mmol). After stirred at r.t. overnight, the reaction mixture was quenched with brine and the resulting mixture was extracted with DCM (x3). The combined organic layers were dried over Na₂SO₄. Solvent was removed under vacuum and the residue was purified by Prep. TLC to afford the title compound. cis-isomer (135 mg, 32% yield). LCMS (ESI) m/z calcd for C₂₃H₃₀N₂O₃: 382.23. Found: 383.24 (M+1)⁺. trans-isomer (44 mg, 10% yield). LCMS (ESI) m/z calcd for C₂₃H₃₀N₂O₃: 382.23. Found: 383.25 (M+1)⁺.

### Example 14

### Preparation of (S)-3-methyl-2-(cis-4-(quinolin-4-yl)cyclohexyl)acetamido) butanamide

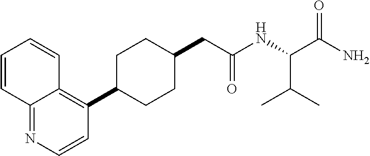 [SMILES: CC(C)[C@H](NC(=O)C[C@H]1CC[C@@H](c2ccnc3ccccc32)CC1)C(N)=O]

A mixture of methyl (2-(cis-4-(quinolin-4-yl)cyclohexyl)acetyl)-L-valinate (130 mg, 0.354 mmol) and 2 M NH₃ in MeOH (3 mL) was stirred at 90° C. for 2 days. The reaction mixture was concentrated and the residue was purified by column chromatography on silica gel to afford the title compound (43 mg, 34% yield). ¹H NMR (400 MHz, DMSO) 6 8.86 (d, J=4.6 Hz, 1H), 8.22 (d, J=8.2 Hz, 1H), 8.02 (dd, J=8.4, 0.9 Hz, 1H), 7.86 (d, J=9.1 Hz, 1H), 7.78-7.71 (m, 1H), 7.66-7.58 (m, 1H), 7.52 (d, J=4.6 Hz, 1H), 7.37 (s, 1H), 6.98 (s, 1H), 4.18 (dd, J=9.1, 6.7 Hz, 1H), 3.44-3.36 (m, 1H), 2.61-2.54 (m, 1H), 2.33-2.22 (m, 2H), 2.00-1.87 (m, 2H), 1.83-1.59 (m, 7H), 0.85 (m, J=6.8 Hz, 6H). LCMS (ESI) m/z calcd for C₂₂H₂₉N₃O₂: 367.23. Found: 368.27 (M+1)⁺.

### Example 15

### Preparation of (S)-3-methyl-2-(trans-4-(quinolin-4-yl)cyclohexyl)acetamido) butanamide

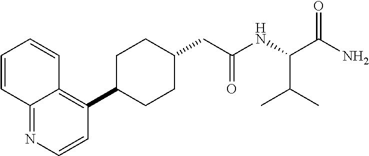 [SMILES: CC(C)[C@H](NC(=O)C[C@H]1CC[C@H](c2ccnc3ccccc32)CC1)C(N)=O]

A mixture of methyl (2-(trans-4-(quinolin-4-yl)cyclohexyl)acetyl)-L-valinate (44 mg, 0.12 mmol) and 2 M NH₃ in MeOH (2 mL) was stirred at 90° C. for 2 days. The reaction mixture was concentrated and the residue was purified by column chromatography on silica gel to afford the title compound (6 mg, 15% yield). ¹H NMR (400 MHz, DMSO) δ 8.81 (d, J=4.6 Hz, 1H), 8.22 (d, J=8.1 Hz, 1H), 8.02 (d, J=8.4 Hz, 1H), 7.78-7.71 (m, 2H), 7.64-7.60 (m, 1H), 7.42 (d, J=4.6 Hz, 1H), 7.34 (s, 1H), 6.98 (s, 1H), 4.15 (dd, J=9.0, 6.7 Hz, 1H), 3.39-3.34 (m, 1H), 2.21-2.12 (m, 2H), 2.00-1.81 (m, 6H), 1.62-1.52 (m, 2H), 1.34-1.25 (m, 2H), 0.91-0.77 (m, J=6.5 Hz, 6H). LCMS (ESI) m/z calcd for C₂₂H₂₉N₃O₂: 367.23. Found: 368.31 (M+1)⁺.

### Example 16

### Preparation of (R)-3-methyl-2-(241s,4S)-4-(quinolin-4-yl)cyclohexyl)acetamido) butanamide

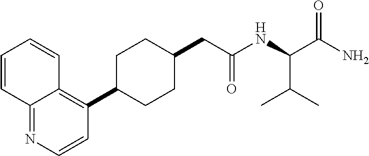 [SMILES: CC(C)[C@@H](NC(=O)C[C@H]1CC[C@@H](c2ccnc3ccccc32)CC1)C(N)=O]

The title compound was prepared from 2-(4-(quinolin-4-yl)cyclohexyl)acetic acid (180 mg, 0.67 mmol) and (R)-2-amino-3-methylbutanamide according to the procedure described for the synthesis of (S)-3-methyl-2-(trans-4-(quinolin-4-yl)cyclohexyl)acetamido) butanamide (scheme 4). ¹H NMR (400 MHz, DMSO) δ 8.87 (d, J=4.5 Hz, 1H), 8.23 (d, J=8.4 Hz, 1H), 8.03 (d, J=8.3 Hz, 1H), 7.86 (d, J=9.0 Hz, 1H), 7.76 (t, J=7.5 Hz, 1H), 7.63 (t, J=7.6 Hz, 1H), 7.53 (d, J=4.5 Hz, 1H), 7.38 (s, 1H), 6.99 (s, 1H), 4.22-4.13 (m, 1H), 3.44-3.38 (m, 1H), 2.61-2.55 (m, 1H), 2.34 -2.24 (m, 2H), 2.00-1.89 (m, 2H), 1.82-1.60 (m, 7H), 0.90-0.81 (m, 6H). LCMS (ESI) m/z calcd for C₂₂H₂₉N₃O₂: 367.23. Found: 368.32 (M+1)⁺.

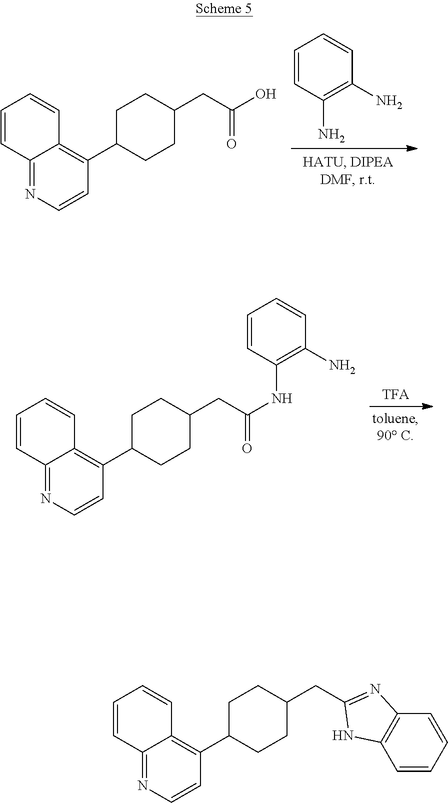

### Preparation of N-(2-aminophenyl)-2-(4-(quinolin-4-0)cyclohexyl)acetamide

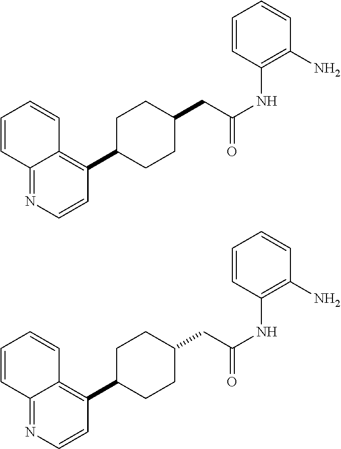 [SMILES: Nc1ccccc1NC(=O)C[C@H]1CC[C@@H](c2ccnc3ccccc32)CC1 ; Nc1ccccc1NC(=O)C[C@H]1CC[C@H](c2ccnc3ccccc32)CC1]

To a stirred solution of 2-(4-(quinolin-4-yl)cyclohexyl)acetic acid (300 mg, 1.11 mmol) and benzene-1,2-diamine (242 mg, 2.24 mmol) in DMF (5 mL) was added DIPEA (0.60 mL, 3.36 mmol) followed by HATU (851 mg, 2.24 mmol). After stirred at r.t. overnight, the reaction mixture was quenched with brine and the resulting mixture was extracted with DCM (x3). The combined organic layers were dried over Na₂SO₄. Solvent was removed under vacuum and the residue was purified by Prep. TLC to afford the title compound. cis-isomer (170 mg, 43% yield). LCMS (ESI) m/z calcd for C₂₃H₂₅N₃O: 359.20. Found: 360.44 (M+1)⁺. trans-isomer (100 mg, 25% yield). LCMS (ESI) m/z calcd for C₂₃H₂₅N₃O: 359.20. Found: 360.41 (M+1)⁺.

### Example 17

### Preparation of 4-(4-cis-((1 H-benzoidlimidazol-2-14)methtincyclohexyl)quinoline

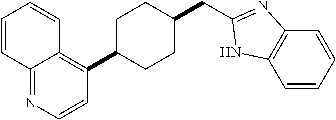 [SMILES: c1ccc2[nH]c(C[C@H]3CC[C@@H](c4ccnc5ccccc54)CC3)nc2c1]

A mixture of N-(2-aminophenyl)-2-(4-cis--(quinolin-4-yl)cyclohexyl)acetamide (170 mg, 0.47 mmol), TFA (3 mL) and toluene (3 mL) was heated to 90° C. After stirred at this temperature overnight, the reaction mixture was concentrated and the residue was was purified by Prep. HPLC to afford the title compound (84 mg, 52% yield). ¹H NMR (400 MHz, CDCl₃) δ 8.81 (d, J=5.0 Hz, 1H), 8.15 (d, J=8.4 Hz, 1H), 8.05 (d, J=8.5 Hz, 1H), 7.77-7.72 (m, 1H), 7.69-7.56 (m, 4H), 7.38-7.31 (m, 2H), 3.32 (d, J=8.2 Hz, 3H), 2.64-2.58 (m, 1H), 1.85-1.57 (m, 8H). Proton of nitrogen in the imidazole ring was not observed. LCMS (ESI) m/z calcd for C₂₃H₂₃N₃: 341.19. Found: 342.40 (M+1)⁺.

The following compounds in Table 2 were prepared similarly to the above procedures using appropriate carboxylic acid and appropriate diamine.

TABLE 2

| | | Exact | M + 1 |
|---|---|---|---|
| Example | Structure | mass | observed |
| 18 | 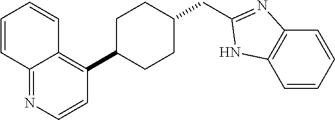 [SMILES: c1ccc2[nH]c(C[C@H]3CC[C@H](c4ccnc5ccccc54)CC3)nc2c1] | 341.19 | 342.40 |
| 19 | 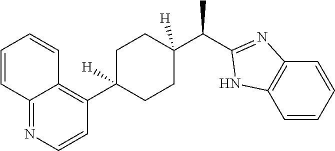 [SMILES: C[C@@H](c1nc2ccccc2[nH]1)[C@H]1CC[C@@H](c2ccnc3ccccc32)CC1] | 355.20 | 356.62 |
| 20 | 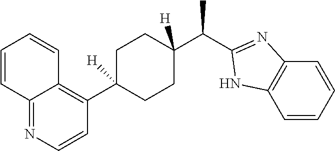 [SMILES: C[C@@H](c1nc2ccccc2[nH]1)[C@H]1CC[C@H](c2ccnc3ccccc32)CC1] | 355.20 | 356.47 |
| 21 | 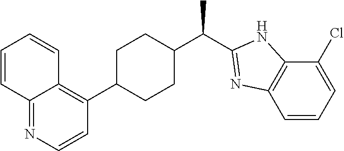 [SMILES: C[C@@H](c1nc2cccc(Cl)c2[nH]1)C1CCC(c2ccnc3ccccc23)CC1] | 389.17 | 388.42/ 390.44 |

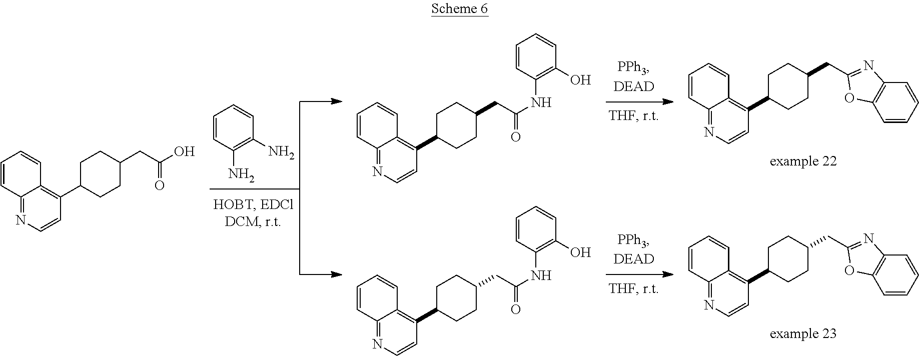 [SMILES: O=C(O)CC1CCC(c2ccnc3ccccc23)CC1 ; Nc1ccccc1N ; O=C(C[C@H]1CC[C@@H](c2ccnc3ccccc32)CC1)Nc1ccccc1O ; O=C(C[C@H]1CC[C@H](c2ccnc3ccccc32)CC1)Nc1ccccc1O ; c1ccc2oc(C[C@H]3CC[C@@H](c4ccnc5ccccc54)CC3)nc2c1 ; c1ccc2oc(C[C@H]3CC[C@H](c4ccnc5ccccc54)CC3)nc2c1]

### Preparation of N-(2-hydroxyphenyI)-2-(cis-4-(quinolin-4-yl)cyclohexyl)acetamide and N-(2-hydroxyphenyl)-2-(trans-4-(quinolin-4-yl)cyclohexyl) acetamide

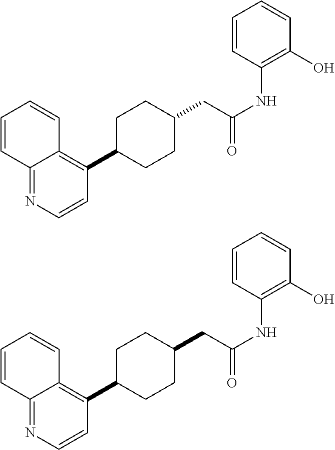 [SMILES: O=C(C[C@H]1CC[C@H](c2ccnc3ccccc32)CC1)Nc1ccccc1O ; O=C(C[C@H]1CC[C@@H](c2ccnc3ccccc32)CC1)Nc1ccccc1O]

To a stirred solution of 2-(4-(quinolin-4-yl)cyclohexyl)acetic acid (300 mg, 1.11 mmol) and 2-aminophenol (240 mg, 2.24 mmol), HOBt (315 mg, 2.24 mmol) in DCM (5 mL) was added DIPEA (0.40 mL, 2.24 mmol) followed by EDCl (435 mg, 2.24 mmol). After stirred at r.t. overnight, the reaction mixture was quenched with brine and the resulting mixture was extracted with DCM (x3). The combined organic layers were dried over Na₂SO₄. Solvent was removed under vacuum and the residue was purified by Prep. TLC to afford the title compound. cis-isomer (140 mg, 35% yield). LCMS (ESI) m/z calcd for C₂₃H₂₄N₂O₂: 360.18. Found: 361.35 (M+1)⁺. trans-isomer (85 mg, 21% yield). LCMS (ESI) m/z calcd for C₂₃H₂₄N₂O₂: 360.18. Found: 361.33 (M+1)⁺.

### Example 22

### Preparation of 2-(((1s,4s)-4-(quinolin-4-yl)cyclohexyl)methyl)benzo[d]oxazole

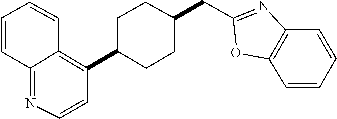 [SMILES: c1ccc2oc(C[C@H]3CC[C@@H](c4ccnc5ccccc54)CC3)nc2c1]

To a mixture of N-(2-hydroxyphenyl)-2-(cis-4-(quinolin-4-y0cyclohexyl)acetamide (140 mg, 0.39 mmol) and PPh₃ (231 mg, 0.88 mmol) in dry THF (15 ml), DEAD (0.14 mL, 0.88 mmol) was added dropwise. After stirred at room temperature overnight, the reaction mixture was concentrated and the residue was purified by Prep. HPLC to afford the title compound (59 mg, 44% yield). ¹H NMR (400 MHz, CDCl₃) δ 8.88 (d, J=4.5 Hz, 1H), 8.11 (dd, J=21.2, 8.0 Hz, 2H), 7.73-7.67 (m, 2H), 7.59-7.54 (m, 1H), 7.52-7.48 (m, 1H), 7.38 (d, J=4.6 Hz, 1H), 7.34-7.29 (m, 2H), 3.49-3.38 (m, 1H), 3.14 (d, J=7.9 Hz, 2H), 2.72-2.60 (m, 1H), 1.94-1.82 (m, 8H). LCMS (ESI) m/z calcd for C₂₃H₂₂N₂O: 342.17. Found: 343.46 (M+1)⁺.

### Example 23

### Preparation of 2-(((1 r, 4r)-4-(quinolin-4-yl)cyclohexyl)methyl)benzo[d]oxazole

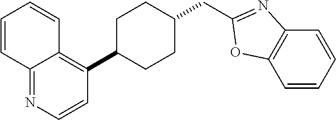 [SMILES: c1ccc2oc(C[C@H]3CC[C@H](c4ccnc5ccccc54)CC3)nc2c1]

The title compound was prepared from N-(2-hydroxyphenyl)-2-(trans-4-(quinolin-4-yl)cyclohexyl) acetamide in 46% yield according to the procedure described above. ¹H NMR (400 MHz, CDCl₃) δ 8.84 (d, J=4.6 Hz, 1H), 8.15-8.06 (m, 2H), 7.74-7.66 (m, 2H), 7.59-7.49 (m, 2H), 7.35-7.29 (m, 2H), 7.28-7.26 (m, 1H), 3.39-3.30 (m, 1H), 2.96 (d, J=6.9 Hz, 2H), 2.20-2.12 (m, 1H), 2.11-2.03 (m, 4H), 1.68-1.62 (m, 2H), 1.51-1.41 (m, 2H). LCMS (ESI) m/z calcd for C₂₃H₂₂N₂O: 342.17. Found: 343.50 (M+1)⁺.

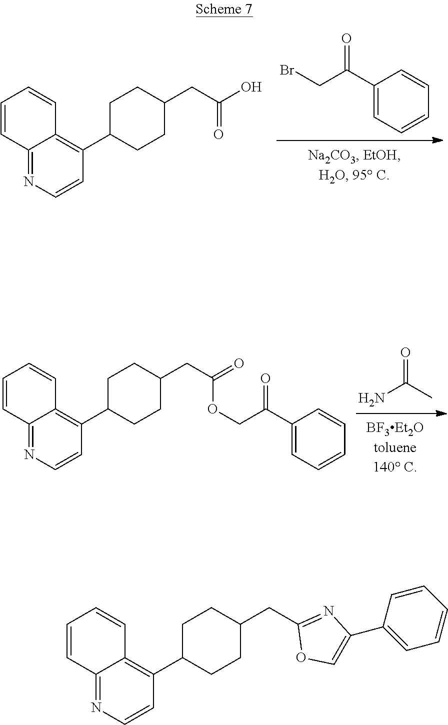

### Preparation of 2-oxo-2-phenylethyl 2-(cis-4-(quinolin-4-yl)cyclohexyl)acetate and 2-oxo-2-phenylethyl 2-(trans-4-(quinolin-4-yl)cyclohexyl) acetate

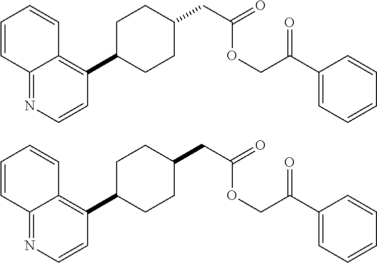

A mixture of 2-(4-(quinolin-4-yl)cyclohexyl)acetic acid (350 mg, 1.3 mmol), 2-bromo-1-phenylethan-1-one (259 mg, 1.3 mmol), Na₂CO₃ (69 mg, 0.65 mmol), H₂O (4 mL) and EtOH (8 mL) was heated to reflux and stirred at this temperature for 2 hours. The reaction mixture was quenched with brine and the resulting mixture was extracted with EtOAc (x3). The combined organic layers were dried over Na₂SO₄. Solvent was removed under vacuum and the residue was purified by Prep. TLC to afford the title compound. cis-isomer (235 mg, 47% yield). LCMS (ESI) m/z calcd for C₂₅H₂₅NO₃: 387.18. Found: 388.45 (M+1)⁺. trans-isomer (120 mg, 24% yield). LCMS (ESI) m/z calcd for C₂₅H₂₅NO₃: 387.18. Found: 388.47 (M+1)⁺.

### Example 24

### Preparation of 4-phenyl-2-((trans-4-(quinolin-4-yl)cyclohexyl)methyl)oxazole

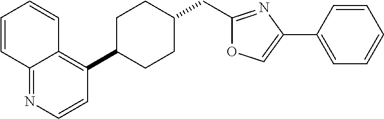 [SMILES: c1ccc(-c2coc(C[C@H]3CC[C@H](c4ccnc5ccccc54)CC3)n2)cc1]

To a solution of 2-oxo-2-phenylethyl 2-(trans-4-(quinolin-4-yl)cyclohexyl) acetate (120 mg, 0.31 mmol), acetamide (92 mg, 1.55 mmol) and toluene (5 ml), BF₃.Et₂O (1 drop) was added dropwise. The mixture was heated at 140° C. for 10 hours. The reaction mixture was partitioned between EtOAc and water. The layers were separated, the aqueous phase was extracted with EtOAc. The combined organic layers were dried over Na₂SO₄ and concentrated to give a residue, which was purified by Prep. HPLC to afford the title compound (31 mg, 27% yield). ¹H NMR (400 MHz, CDCl₃) δ 8.84 (d, J=4.6 Hz, 1H), 8.16-8.05 (m, 2H), 7.86 (s, 1H), 7.81-7.65 (m, 3H), 7.59-7.53 (m, 1H), 7.46-7.36 (m, 2H), 7.33-7.27 (m, 2H), 3.38-3.29 (m, 1H), 2.84 (d, J=6.8 Hz, 2H), 2.11-1.98 (m, 5H), 1.70-1.63 (m, 2H), 1.48-1.38 (m, 2H). LCMS (ESI) m/z calcd for C₂₅H₂₄N₂O: 368.19. Found: 369.41 (M+1)⁺.

The following compounds in Table 3 were prepared similarly to the above procedures using 2-(4-(quinolin-4-yl)cyclohexyl)acetic acid and appropriate a-bromo ketone.

TABLE 3

| | | Exact | M + 1 |
|---|---|---|---|
| Example | structure | mass | observed |
| 25 | 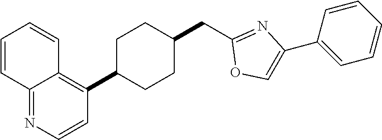 [SMILES: c1ccc(-c2coc(C[C@H]3CC[C@@H](c4ccnc5ccccc54)CC3)n2)cc1] | 368.19 | 369.41 |
| 26 | 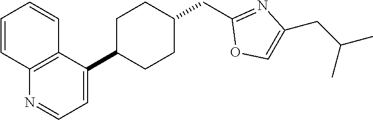 [SMILES: CC(C)Cc1coc(C[C@H]2CC[C@H](c3ccnc4ccccc43)CC2)n1] | 348.22 | 349.31 |
| 27 | 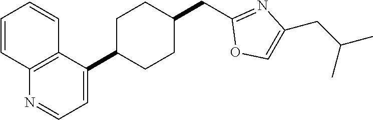 [SMILES: CC(C)Cc1coc(C[C@H]2CC[C@@H](c3ccnc4ccccc43)CC2)n1] | 348.22 | 349.39 |
| 28 | 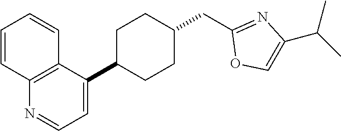 [SMILES: CC(C)c1coc(C[C@H]2CC[C@H](c3ccnc4ccccc43)CC2)n1] | 334.20 | 335.30 |
| 29 | 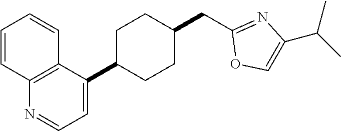 [SMILES: CC(C)c1coc(C[C@H]2CC[C@@H](c3ccnc4ccccc43)CC2)n1] | 334.20 | 335.37 |

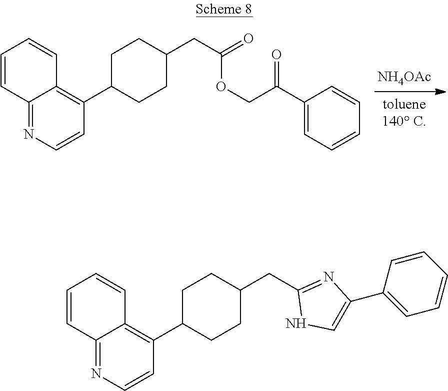 [SMILES: O=C(CC1CCC(c2ccnc3ccccc23)CC1)OCC(=O)c1ccccc1 ; c1ccc(-c2c[nH]c(CC3CCC(c4ccnc5ccccc45)CC3)n2)cc1 ; "NH4OAc"]

### Example 30

### Preparation of 4-(trans-4-((4-ohenv1-1H-imidazol-2-yl)methyl)cyclohexyl)quinoline

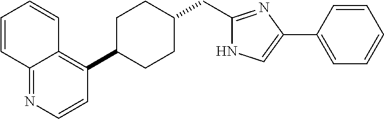 [SMILES: c1ccc(-c2c[nH]c(C[C@H]3CC[C@H](c4ccnc5ccccc54)CC3)n2)cc1]

A mixture of 2-oxo-2-phenylethyl 2-(trans-4-(quinolin-4-yl)cyclohexyl) acetate (109 mg, 0.28 mmol), NH₄OAc (440 mg, 5.6 mmol) and toluene (3 ml) was heated at 140° C. for 15 hours. The reaction mixture was partitioned between EtOAc and water. The layers were separated, the aqueous phase was extracted with EtOAc. The combined organic layers were dried over Na₂SO₄ and concentrated to give a residue, which was purified by Prep. HPLC to afford the title compound (32 mg, 31% yield). ¹H NMR (400 MHz, DMSO) δ 12.12 (br, 1H), 8.81 (d, J=4.6 Hz, 1H), 8.22 (d, J=8.0 Hz, 1H), 8.01 (dd, J=8.4, 0.9 Hz, 1H), 7.80-7.68 (m, 3H), 7.66-7.58 (m, 1H), 7.47 (s, 1H), 7.40 (d, J=4.6 Hz, 1H), 7.38-7.31 (m, 2H), 7.21-7.14 (m, 1H), 3.43-3.38 (m, 1H), 2.65 (d, J=6.6 Hz, 2H), 1.97-1.82 (m, 5H), 1.64-1.53 (m, 2H), 1.42-1.33 (m, 2H). LCMS (ESI) m/z calcd for C₂₅H₂₅N₃: 367.20. Found: 368.50 (M+1)⁺.

The following compounds in Table 4 were prepared similarly to the above procedures using appropriate carboxylic acid and appropriate α-bromo ketone.

TABLE 4

| | | Exact | M + 1 |
|---|---|---|---|
| Example | structure | mass | observed |
| 31 | 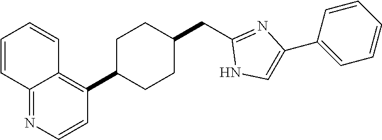 [SMILES: c1ccc(-c2c[nH]c(C[C@H]3CC[C@@H](c4ccnc5ccccc54)CC3)n2)cc1] | 367.20 | 368.56 |
| 32 | 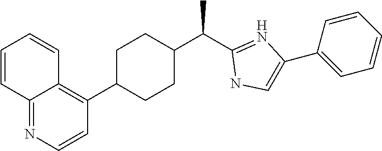 | 381.22 | 382.68 |
| 33 | 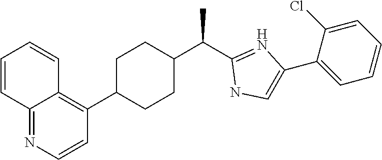 | 415.18 | 416.29/ 418.31 |
| 34 | 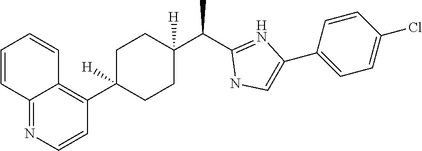 | 415.18 | 416.24/ 418.21 |
| 35 | 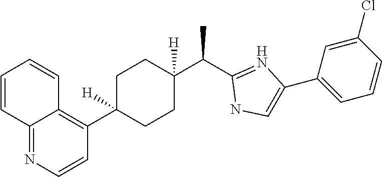 | 415.18 | 416.36/ 418.40 |

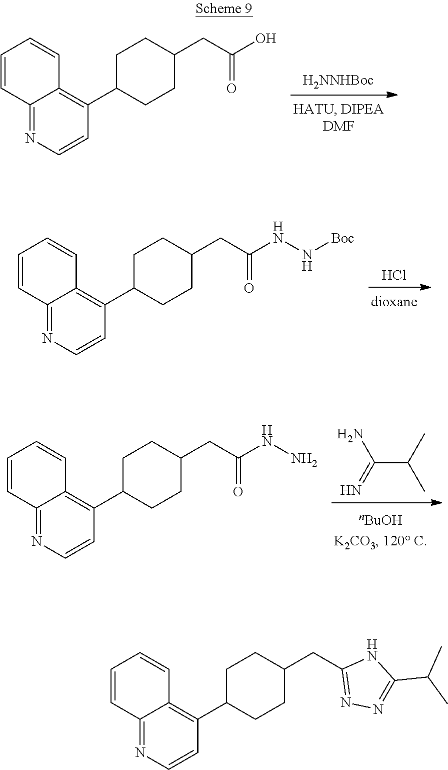

### Preparation of tent-butyl 2-(2-(4-(quinolin-4-yl)cyclohexyl)acetyl)hydrazine-1-carboxylate

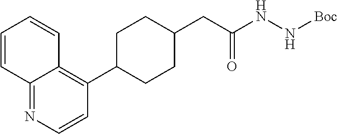 [SMILES: CC(C)(C)OC(=O)NNC(=O)CC1CCC(c2ccnc3ccccc23)CC1]

To a stirred solution of 2-(4-(quinolin-4-yl)cyclohexyl)acetic acid (300 mg, 1.11 mmol) and tert-butyl hydrazinecarboxylate (220 mg, 1.67 mmol) in DMF (5 mL) was added DIPEA (0.60 mL, 3.33 mmol) followed by HATU (464 mg, 1.22 mmol). After stirred at r.t. overnight, the reaction mixture was quenched with brine and the resulting mixture was extracted with DCM (x3). The combined organic layers were dried over Na₂SO₄. Solvent was removed under vacuum and the residue was purified by column chromatography to afford the title compound (420 mg, 98% yield). LCMS (ESI) m/z calcd for C₂₂H₂₉N₃O₃: 383.22. Found: 384.36 (M+1)⁺.

### Preparation of 2-(4-(quinolin-4-yl)cyclohexyl)acetohydrazide

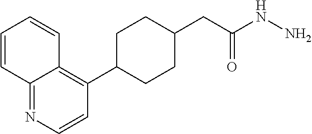 [SMILES: NNC(=O)CC1CCC(c2ccnc3ccccc23)CC1]

To a solution of tert-butyl 4-(1-(4-fluorobenzamido)-3-methylbutyl)piperidine-1-carboxylate (420 g, 1.10 mmol) in DCM (3 mL), was added 4 M HCl in dioxane (4 mL) dropwise. After stirred at r.t. for 2 h, the reaction mixture was concentrated to to afford a hydrochloride salt of the title compound (340 mg, 97% yield), which was used in the following step without purification. LCMS (ESI) m/z calcd for C₁₇H₂₁N₃O: 283.17. Found: 284.28 (M+1)⁺.

### Example 36 and Example 37

### Preparation of 4-(cis-4-((5-isopropyl-4H-1,2,4-triazol-3-yl)methyl)cyclohexyl) quinoline and 4-(trans-4-((5-isopropyl-4H-1,2,4-triazol-3-yl)methyl) cyclohexyl)quinoline

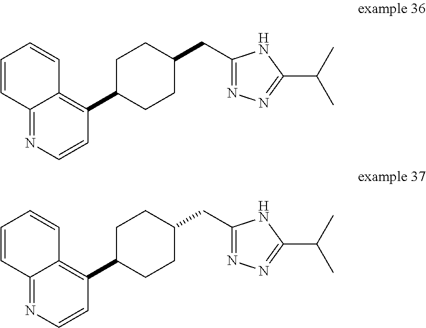 [SMILES: CC(C)c1nnc(C[C@H]2CC[C@@H](c3ccnc4ccccc43)CC2)[nH]1 ; CC(C)c1nnc(C[C@H]2CC[C@H](c3ccnc4ccccc43)CC2)[nH]1]

A mixture of 2-(4-(quinolin-4-yl)cyclohexyl)acetohydrazide (340 mg, 1.06 mmol), isobutyrimidamide (194 mg, 1.59 mmol), K₂CO₃ (585 mg, 4.24 mmol) and n-BuOH (5 mL) was stirred at 120° C. for 8 hours. The reaction mixture was partitioned between water and EtOAc and the layers were separated. The organics were washed sequentially with water and brine, and dried over Na₂SO₄. Filtration and concentration in vacuum gave a crude product, which was purified by Prep. TLC to afford example 36; 4-(cis-4-((5-isopropyl-4H-1,2,4-triazol-3-yl)methyl)cyclohexyl) quinoline (14 mg, 4% yield). ¹H NMR (400 MHz, DMSO) δ 13.20 (br, 1H), 8.85 (d, J=4.5 Hz, 1H), 8.22 (d, J=8.2 Hz, 1H), 8.03 (dd, J=8.4, 0.9 Hz, 1H), 7.78-7.71 (m, 1H), 7.66-7.59 (m, 1H), 7.51 (d, J=4.6 Hz, 1H), 3.46-3.40 (m, 1H), 2.99-2.88 (m, 1H), 2.81 (d, J=6.8 Hz, 2H), 2.33-2.25 (m, 1H), 1.90-1.58 (m, 8H), 1.23 (d, J=6.9 Hz, 6H). (ESI) m/z calcd for C₂₁H₂₆N₄: 334.22. Found: 335.25 (M+1)⁺. Example 37; 4-(trans-4-((5-isopropyl-4H-1,2,4-triazol-3-yl)methyl) cyclohexyl)quinoline (7 mg, 2% yield). ¹H NMR (400 MHz, DMSO) δ 13.19 (s, 1H), 8.81 (d, J=4.3 Hz, 1H), 8.22 (d, J=8.3 Hz, 1H), 8.01 (d, J=8.3 Hz, 1H), 7.80-7.69 (m, 1H), 7.68-7.57 (m, 1H), 7.41 (d, J=4.4 Hz, 1H), 3.44-3.38 (m, 1H), 3.01-2.87 (m, 1H), 2.65-2.54 (m, 2H), 2.03-1.78 (m, 5H), 1.67-1.51 (m, 2H), 1.39-1.29 (m, 2H), 1.23 (d, J=6.5 Hz, 6H). (ESI) m/z calcd for C₂₁H₂₆N₄: 334.22. Found: 335.29 (M+1)⁺.

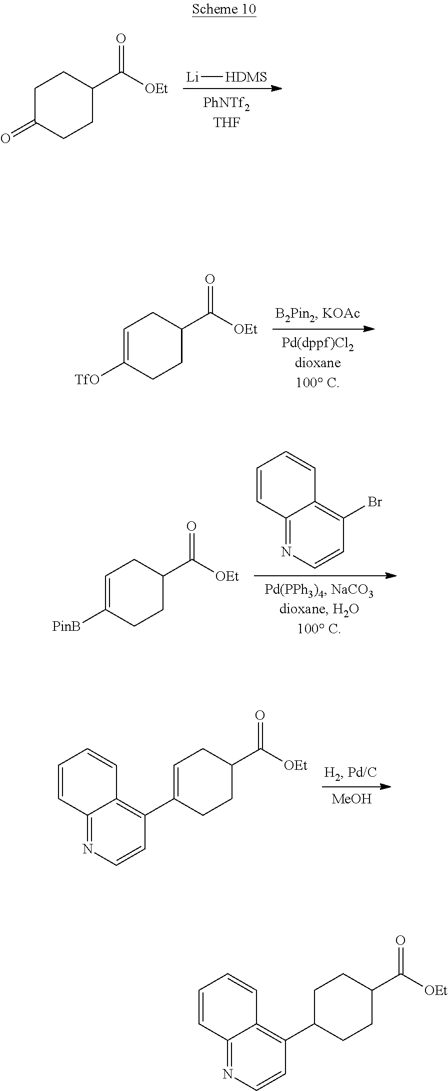

### Preparation of ethyl 4-(((trifluoromethyl)sulfonyl)oxy)cyclohex-3-ene-1-carboxylate

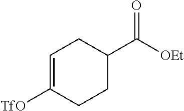 [SMILES: CCOC(=O)C1CC=C(OS(=O)(=O)C(F)(F)F)CC1]

To a solution of ethyl-4-cyclohexanonecarboxylate (10.0 g, 58.8 mmol) in THF (220 ml) was added a 1M solution of LiHMDS in THF (62 ml, 62 mmol) at −78° C. Stirring for 1 h was followed by addition of a solution of N-phenyl-bis(trifluoromethanesulfonimide) (22 g, 62 mmol) in THF (30 ml). The cooling bath was removed 30 minutes after completed addition, and the reaction mixture was stirred for 12 h at room temperature. The mixture was quenched with 1 M aqueous sodium hydrogen sulfate solution (62 ml, 62 mmol). The solvent was removed by rotary evaporation. The resulting mixture was extracted with EtOAc. The organics were washed sequentially with water and brine, and dried over Na₂SO₄. Filtration and concentration in vacuum gave a crude product, which was purified by flash chromatography (silica gel, 0-10% EtOAc in PE) to afford the title compound (15 g, 84% yield). (ESI) m/z calcd for C₁₀H₁₃F₃O₅S: 302.04. Found: 303.37 (M+1)⁺.

### Preparation of ethyl 4-(4,4,5,5-tetramethyl-1,3,2-dioxaborolan-2-yl)cyclohex-3-ene-1-carboxylate

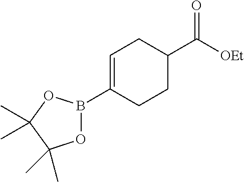 [SMILES: CCOC(=O)C1CC=C(B2OC(C)(C)C(C)(C)O2)CC1]

A mixture of ethyl 4-((((trifluoromethyl)sulfonyl)oxy)cyclohex-3-ene-1-carboxylate (15.7 g, 52 mmol), potassium acetate (15.3 g, 156 mmol), bis(pinacolato)diboron (19.8 g, 78 mmol), dichloro(1,1′-bis(diphenylphosphino)ferrocene)palladium(II) (2.12 g, 2.6 mmol) in 1,4-dioxane (200 ml) was stirred at 90° C. under nitrogen atmosphere for 18 h. The reaction mixture was partitioned between ethyl acetate and water. The layers were separated. The organic layer was washed with brine, dried over anhydrous sodium sulfate and concentrated to dryness. Flash-chromatography on silica gel with n-heptane/ethyl acetate as eluent gave the title compound (13.9 g, 95%) as a light yellow oil. (ESI) m/z calcd for C₁₅H₂₅BO₄: 280.18. Found: 281.35 (M+1)⁺.

### Preparation of ethyl 4-(quinolin-4-yl)cyclohex-3-ene-1-carboxylate

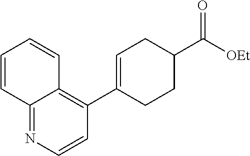 [SMILES: CCOC(=O)C1CC=C(c2ccnc3ccccc23)CC1]

To a suspension of ethyl 4-(4,4,5,5-tetramethyl-1,3,2-dioxaborolan-2-y0cyclohex-3-ene-1-carboxylate (13.4 g, 47.8 mmol), 4-bromoquinoline (9.9 g, 47.8 mmol), Pd(PPh₃)₄ (5.5 g, 4.8 mmol) and in dioxane (100 mL) and water (38 mL), was added sodium carbonate (15.2 g, 143 mmol) and the mixture was stirred at 100° C. under nitrogen atmosphere for 14 hours. After the reaction mixture was cooled to room temperature, this was partitioned between water and EtOAc and the layers were separated. The organics were washed sequentially with water and brine, and dried over Na₂SO₄. Filtration and concentration in vacuum gave a crude product, which was purified by flash chromatography to afford the title compound (9.2 g, 69% yield). (ESI) m/z calcd for C₁₈H₁₉NO₂: 281.14. Found: 282.54 (M+1)⁺.

### Preparation of ethyl cis-4-(quinolin-4-yl)cyclohexane-1-carboxylate and ethyl trans-4-(quinolin-4-v1)cyclohexane-1-carboxylate

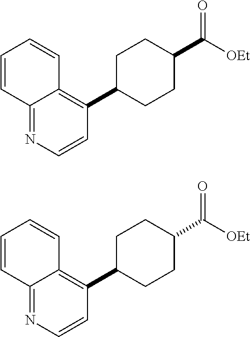 [SMILES: CCOC(=O)[C@H]1CC[C@@H](c2ccnc3ccccc32)CC1 ; CCOC(=O)[C@H]1CC[C@H](c2ccnc3ccccc32)CC1]

A mixture of ethyl 4-(quinolin-4-yl)cyclohex-3-ene-1-carboxylate (9.2 g, 32.7 mmol) and 10% Pd/C (4.6 g) in EtOAc (50 mL) was stirred at room temperature under H2 atmosphere (15 psi) overnight. The resulting mixture was filtered through a pad of Celite and the filtrate was concentrated under reduced pressure to give the crude product which was purified by flash chromatography (silica gel, 0-50% EtOAc in PE) to afford the title compound, cis-isomer (3.0 g, 32% yield) as a pale solid. ¹H NMR (400 MHz, CDCl₃) δ 8.84 (d, J=4.6 Hz, 1H), 8.15-8.04 (m, 2H), 7.74-7.65 (m, 1H), 7.59-7.52 (m, 1H), 7.27 (d, J=3.4 Hz, 1H), 4.21 (q, J=7.1 Hz, 2H), 3.41-3.30 (m, 1H), 2.84-2.78 (m, 1H), 2.41-2.31 (m, 2H), 1.97-1.87 (m, 2H), 1.86-1.71 (m, 4H), 1.30 (t, J=7.1 Hz, 3H). (ESI) m/z calcd for C₁₈H₂₁NO₂: 283.16. Found: 284.33 (M+1)⁺. trans-isomer (0.90 g, 10% yield) as a pale solid. ¹H NMR (400 MHz, CDCl₃) δ 8.85 (d, J=4.6 Hz, 1H), 8.13 (d, J=8.4 Hz, 1H), 8.07 (d, J=8.4 Hz, 1H), 7.74-7.67 (m, 1H), 7.61-7.53 (m, 1H), 7.26 (d, J=4.6 Hz, 1H), 4.18 (q, J=7.1 Hz, 2H), 3.41-3.31 (m, 1H), 2.49-2.39 (m, 1H), 2.26-2.16 (m, 2H), 2.16-2.08 (m, 2H), 1.82-1.71 (m, 2H), 1.68-1.56 (m, 2H), 1.33-1.20 (m, 3H). (ESI) m/z calcd for C₁₈H₂₁NO₂: 283.16. Found: 284.37 (M+1)⁺;

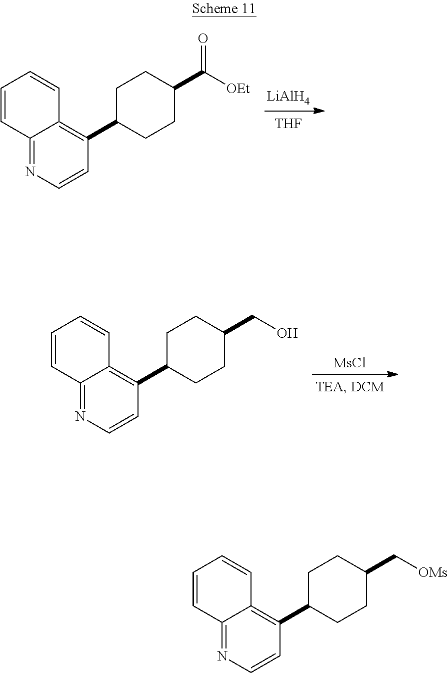

### Preparation of (cis-4-(quinolin-4-yl)cyclohexyl)methanol

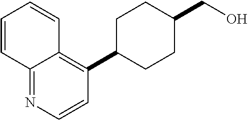 [SMILES: OC[C@H]1CC[C@@H](c2ccnc3ccccc32)CC1]

At 0° C., to a solution of ethyl cis-4-(quinolin-4-yl)cyclohexane-1-carboxylate (2.0 g, 7.1 mmol) in THF was added LiAIH₄ (540 mg, 14.2 mmol) portion wise. After complete addition, the resulting mixture was allowed to warm up to room temperature and stirred for 3 hours. The reaction was quenched by water (0.5 mL), 15% NaOH (1 mL) successively. The solid was filtered off and the filtrate was concentrated in vacuum gave the title compound (1.46 g, 85% yield) as a white solid, which was used in the following step without further purification. (ESI) m/z calcd for C₁₆H₁₉NO: 241.15. Found: 242.37 (M+1)⁺.

### Preparation of (cis-4-(quinolin-4-yl)cyclohexyl)methyl methanesulfonate

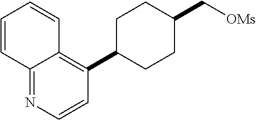 [SMILES: CS(=O)(=O)OC[C@H]1CC[C@@H](c2ccnc3ccccc32)CC1]

At 0° C., to a solution of (cis-4-(quinolin-4-yl)cyclohexyl)methanol (800 mg, 33 mmol) and TEA (0.7 mL, 5.0 mmol) in THF was added MsCl (0.5 mL) drop wise. After complete addition, the resulting mixture was allowed to warm up to room temperature and stirred for 3 hours. The solid was filtered off and the filtrate was concentrated in vacuum. The residue was re-dissolved in EtOAc and the solution was washed with sat. NaHCO₃, brine, dried over Na₂SO₄. Filtration and concentration gave the title compound (1.0 g, 95% yield) as a tan solid, which was used in the following step without further purification. (ESI) m/z calcd for C₁₇H₂₁NO₃S: 319.12. Found: 320.31 (M+1)⁺.

### Preparation of (trans-4-(quinolin-4-yl)cyclohexyl)methyl methanesulfonate

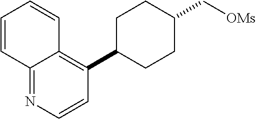 [SMILES: CS(=O)(=O)OC[C@H]1CC[C@H](c2ccnc3ccccc32)CC1]

The title compound was prepared from ethyl trans-4-(quinolin-4-yl)cyclohexane-1-carboxylate according to procedure described above. (ESI) m/z calcd for C₁₇H₂₁NO₃S: 319.12. Found: 320.36 (M+1)⁺.

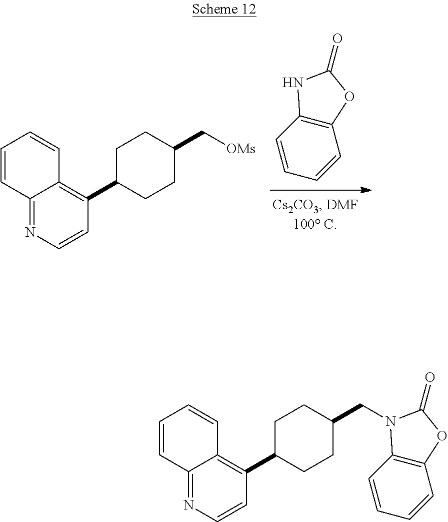

### Example 38

### Preparation of 3-((cis-4-(quinolin-4-yl)cyclohexyl)methyl)benzo[d]oxazol-2(3H)-one

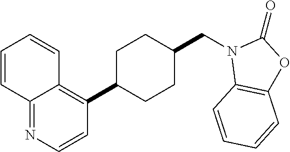 [SMILES: O=c1oc2ccccc2n1C[C@H]1CC[C@@H](c2ccnc3ccccc32)CC1]

A mixture of (cis-4-(quinolin-4-yl)cyclohexyl)methyl methanesulfonate (200 mg, 0.63 mmol), benzo[d]oxazol-2(3H)-one (129 mg, 0.95 mmol), Cs₂CO₃ (620 mg, 1.9 mmol) and DMF (5 mL) was stirred at 100° C. overnight. The reaction mixture was partitioned between water and EtOAc and the layers were separated. The organics were washed sequentially with water and brine, and dried over Na₂SO₄. Filtration and concentration in vacuum gave a crude product, which was purified by Prep. HPLC to afford the title compound (84 mg, 37% yield). ¹H NMR (400 MHz, DMSO) δ 8.88 (d, J=4.5 Hz, 1H), 8.23 (d, J=8.4 Hz, 1H), 8.04 (d, J=8.3 Hz, 1H), 7.75 (t, J=7.6 Hz, 1H), 7.63 (t, J=7.6 Hz, 1H), 7.56 (d, J=4.5 Hz, 1H), 7.38 (dd, J=16.2, 7.8 Hz, 2H), 7.24 (t, J=7.7 Hz, 1H), 7.15 (t, J=7.8 Hz, 1H), 4.02 (d, J=8.0 Hz, 2H), 3.51-3.44 (m, 1H), 2.42-2.36 (m, 1H), 2.00-1.80 (m, 4H), 1.79-1.63 (m, 4H). (ESI) m/z calcd for C₂₃H₂₂N₂O₂: 358.17. Found: 359.29 (M+1)⁺.

The following compounds in Table 5 were prepared according to the above procedures using (4-(quinolin-4-yl)cyclohexyl)methyl methanesulfonate and appropriate material.

TABLE 5

| | | | M + 1 |
|---|---|---|---|
| Example | Structure | Exact mass | observed |
| 39 | 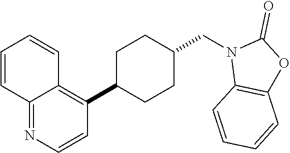 [SMILES: O=c1oc2ccccc2n1C[C@H]1CC[C@H](c2ccnc3ccccc32)CC1] | 358.17 | 359.25 |
| 40 | 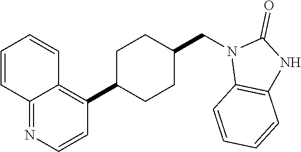 [SMILES: O=c1[nH]c2ccccc2n1C[C@H]1CC[C@@H](c2ccnc3ccccc32)CC1] | 357.18 | 358.30 |
| 41 | 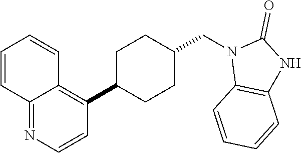 [SMILES: O=c1[nH]c2ccccc2n1C[C@H]1CC[C@H](c2ccnc3ccccc32)CC1] | 357.18 | 358.26 |

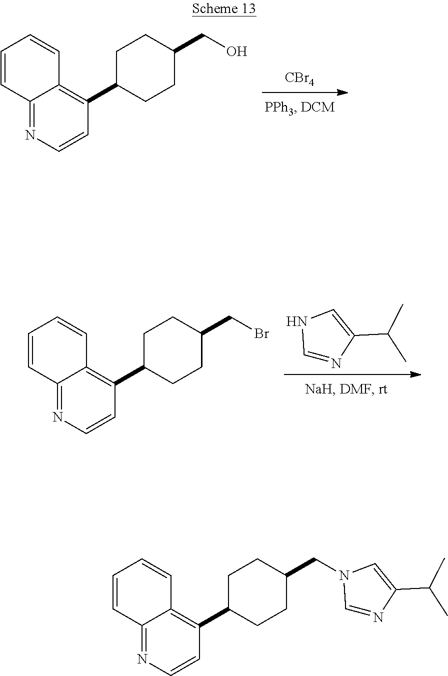

### Preparation of 4-(cis-4-(bromomethyl)cyclohexyl)quinoline

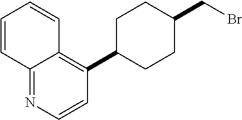 [SMILES: BrC[C@H]1CC[C@@H](c2ccnc3ccccc32)CC1]

At 0° C., to a solution of (cis-4-(quinolin-4-yl)cyclohexyl)methanol (400 mg, 1.66 mmol) and CBr₄ (996 mg, 3.0 mmol) in DCM (10 mL), was added a solution of PPh₃ (894 mg, 3.4 mmol) in DCM (2 mL) drop wise. After stirred at room temperature for 3 hours, the reaction mixture was partitioned between water and EtOAc and the layers were separated. The organics were washed sequentially with brine, dried over Na₂SO₄. Filtration and concentration in vacuum gave a crude product, which was purified by column chromatography on silica gel to afford the title compound (180 mg, 36% yield). (ESI) m/z calcd for C₁₆H₁₈BrN: 303.06. Found: 304.10/306.11 (M/M+2)⁺.

### Example 42

### Preparation of 4-(cis-4-((4-isopropyl-1H-imidazol-1-yl)methyl)cyclohexyl)quinoline

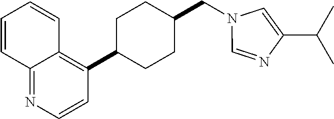 [SMILES: CC(C)c1cn(C[C@H]2CC[C@@H](c3ccnc4ccccc43)CC2)cn1]

At 0° C., to a solution of 4-isopropyl-1 H-imidazole (99 mg, 0.9 mmol) in DMF (5 mL) was added NaH (48 mg, 1.2 mmol). After stirred at 0° C. for 30 min, 4-(cis-4-(bromomethyl) cyclohexyl)quinoline (180 mg, 0.6 mmol) was added and the resulting mixture was stirred at room temperature for 3 hours. The reaction mixture was partitioned between water and EtOAc and the layers were separated. The organics were washed sequentially with water and brine, and dried over Na₂SO₄. Filtration and concentration in vacuum gave a crude product, which was purified by Prep. HPLC to afford the title compound (3.4 g, 2% yield). ¹H NMR (400 MHz, DMSO) δ 8.89-8.84 (m, 1H), 8.22 (d, J=8.3 Hz, 1H), 8.03 (d, J=8.4 Hz, 1H), 7.77-7.72 (m, 1H), 7.65-7.60 (m, 1H), 7.59-7.54 (m, 2H), 6.87 (s, 1H), 4.08 (d, J=8.2 Hz, 2H), 3.47-3.42 (m, 1H), 2.80-2.69 (m, 1H), 2.28-2.18 (m, 1H), 1.89-1.69 (m, 6H), 1.57-1.49 (m, 2H), 1.21-1.14 (m, 6H). (ESI) m/z calcd for C₂₂H₂₇N₃: 333.22. Found: 334.27 (M+1)⁺.

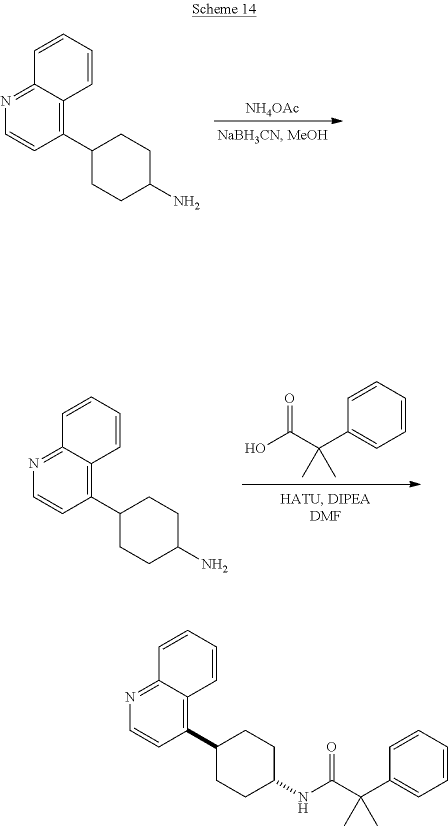 [SMILES: NC1CCC(c2ccnc3ccccc23)CC1 ; NC1CCC(c2ccnc3ccccc23)CC1 ; CC(C)(C(=O)O)c1ccccc1 ; "NH4OAc" ; CC(C)(C(=O)N[C@H]1CC[C@H](c2ccnc3ccccc32)CC1)c1ccccc1]

### Preparation of 4-(quinolin-4-yl)cyclohexan-1-amine

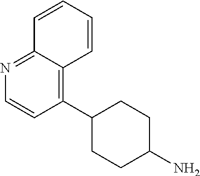 [SMILES: NC1CCC(c2ccnc3ccccc23)CC1]

To a solution of 4-(quinolin-4-yl)cyclohexan-1-one (200 mg, 0.88 mmol) in MeOH (6 mL), was added NH₄OAc (1.37 g, 17.76 mmol) and NaBH₃CN (558 mg, 8.88 mmol) successively. After stirred at r.t. overnight, the reaction was quenched with saturated NH₄Cl aq. solution and extracted with EtOAc. The organic layer was washed with brine, dried over Na₂SO₄, filtered and concentrated to afford the title compound (160 mg, 80% yield), which was used in the following step without further purification. LCMS (ESI) m/z calcd for C₁₅H₁₈N₂: 226.15. Found: 227.15 (M+1)⁺.

### Example 43

### Preparation of 2-methyl-2-phenyl-N-(4-(quinolin-4-yl)cyclohexyl)propanamide

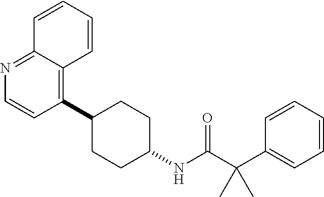 [SMILES: CC(C)(C(=O)N[C@H]1CC[C@H](c2ccnc3ccccc32)CC1)c1ccccc1]

To a solution of 4-(quinolin-4-yl)cyclohexan-1-amine (120 mg, 0.53 mmol) in DMF (3mL), was added 2-methyl-2-phenylpropanoic acid (105 mg, 0.64 mmol), DIPEA (0.28 mL, 1.59 mmol) and HATU (303 mg, 0.80 mmol) successively. After stirred at r.t. overnight, the reaction was diluted with water and extracted with EtOAc. The organic layer was washed with brine, dried over Na₂SO₄, filtered and concentrated to give the crude product which was purified by Prep. HPLC to afford the title compound (34 mg, 17% yield). ¹H NMR (400 MHz, DMSO) δ 8.82 (d, J=4.5 Hz, 1H), 8.17 (d, J=8.4 Hz, 1H), 8.02 (d, J=8.3 Hz, 1H), 7.77-7.71 (m, 1H), 7.65-7.59 (m, 1H), 7.44 (d, J=4.5 Hz, 1H), 7.37-7.31 (m, 4H), 7.26-7.19 (m, 1H), 7.14 (d, J=7.9 Hz, 1H), 3.82-3.69 (m, 1H), 3.32-3.28 (m, 1H), 1.94-1.83 (m, 4H), 1.71-1.61 (m, 2H), 1.57-1.49 (m, 2H), 1.46 (s, 6H). LCMS (ESI) m/z calcd for C₂₅H₂₈N₂O: 372.22. Found: 373.23 (M+1)⁺.

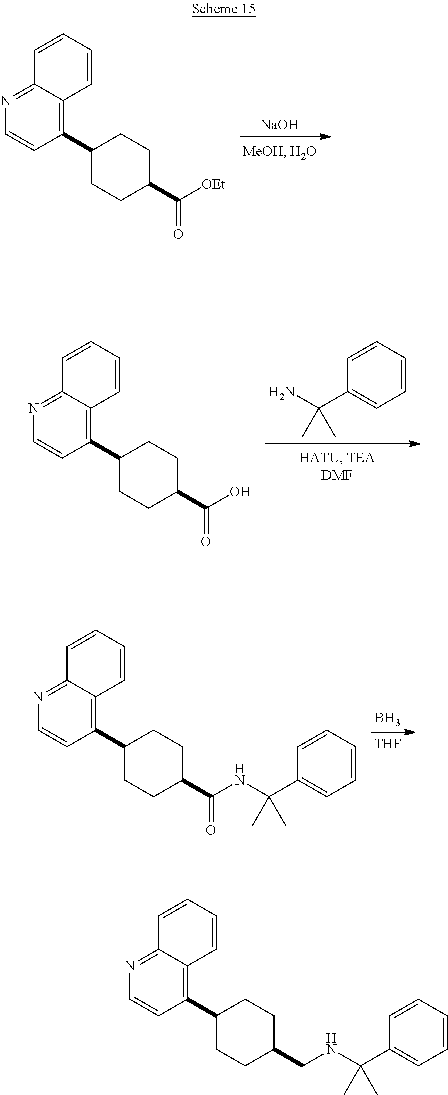

### Preparation of 2-(cis-4-(quinolin-4-yl)cyclohexyl)acetic acid

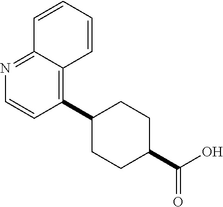 [SMILES: O=C(O)[C@H]1CC[C@@H](c2ccnc3ccccc32)CC1]

To a solution of ethyl 4-(quinolin-4-yl)cyclohexane-1-carboxylate (400 mg, 1.41 mmol) in MeOH (5 mL) was added 1N NaOH aq. (5.6 mL). After stirred at 25° C. overnight, the resulting mixture was neutralized with 1N HCl and extracted with EtOAc. The organic layer was washed with brine, dried over Na₂SO₄, filtered and concentrated to give the title compound (340 mg, 95% yield), which was used in the following step without further purification. LCMS (ESI) m/z calcd for C₁₆H₁₇NO₂: 255.13. Found: 256.33 (M+1)⁺.

### Example 44

### Preparation of 2-methyl-2-phenyl-N-(cis-4-(quinolin-4-yl)cyclohexyl)propanamide

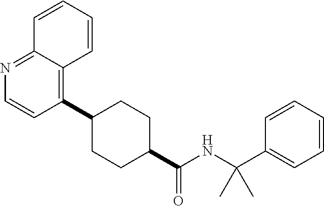 [SMILES: CC(C)(NC(=O)[C@H]1CC[C@@H](c2ccnc3ccccc32)CC1)c1ccccc1]

To a solution of 2-phenylpropan-2-amine (32 mg, 0.24 mmol) in DMF (1 mL), was added 2-(cis-4-(quinolin-4-yl)cyclohexyl)acetic acid (50 mg, 0.20 mmol), TEA (40 mg, 0.39 mmol) and HATU (112 mg, 0.29 mmol) successively. After stirred at r.t. overnight, the reaction was diluted with water and extracted with EtOAc. The organic layer was washed with brine, dried over Na₂SO₄, filtered and concentrated to give the crude product which was purified by Prep. HPLC to afford the title compound (34 mg, 46% yield). ¹H NMR (400 MHz, DMSO) δ 8.79 (d, J=4.5 Hz, 1H), 8.21 (d, J=8.3 Hz, 1H), 8.01 (d, J=8.3 Hz, 1H), 7.91 (s, 1H), 7.77-7.70 (m, 1H), 7.65-7.58 (m, 1H), 7.37-7.32 (m, 2H), 7.30-7.23 (m, 3H), 7.18-7.13 (m, 1H), 3.43-3.37 (m, 1H), 2.70-2.64 (m, 1H), 2.06 (d, J=11.2 Hz, 2H), 1.95-1.86 (m, 2H), 1.82-1.69 (m, 4H), 1.56 (s, 6H). LCMS (ESI) m/z calcd for C₂₅H₂₅N₂O: 372.22. Found: 373.30 (M+1)⁺.

### Example 45

### Preparation of 2-phenyl-N-((cis-4-(quinolin-4-yl)cyclohexyl)methyl)propan-2-amine

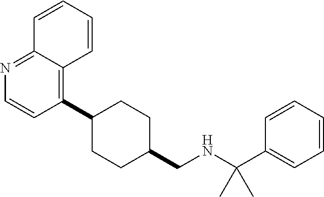 [SMILES: CC(C)(NC[C@H]1CC[C@@H](c2ccnc3ccccc32)CC1)c1ccccc1]

To a solution of 2-methyl-2-phenyl-N-(cis-4-(quinolin-4-yl)cyclohexyl)propanamide (150 mg, 0.40 mmol) in THF was added BH₃.THF (0.80 mL, 0.80 mmol). After stirred at reflux for 70 min, the reaction mixture was quenched with MeOH and conc. HCl. The resulting mixture was neutralized to pH 7 with sat. NaHCO₃ aqueous solution and extracted with EtOAc. The organic layer was washed with brine, dried over Na₂SO₄, filtered and concentrated to give the crude product which was purified by Prep. HPLC to afford the title compound (29 mg, 20% yield). ¹H NMR (400 MHz, DMSO) δ 8.78 (d, J=4.5 Hz, 1H), 8.24 (s, 1H), 8.17 (d, J=8.3 Hz, 1H), 8.00 (d, J=8.3 Hz, 1H), 7.76-7.70 (m, 1H), 7.64-7.57 (m, 1H), 7.53 (d, J=7.3 Hz, 2H), 7.38-7.30 (m, 2H), 7.27-7.17 (m, 2H), 3.39-3.32 (m, 1H), 2.38 (d, J=6.9 Hz, 2H), 1.89-1.78 (m, 3H), 1.77-1.67 (m, 2H), 1.65-1.56 (m, 2H), 1.53-1.34 (m, 8H). LCMS (ESI) m/z calcd for C₂₅H₃₀N₂: 358.24. Found: 359.48 (M+1)⁺.

### Example 46

### Preparation of 2-methyl-2-phenyl-N-(cis-4-(quinolin-4-yl)cyclohexyl)propanamide

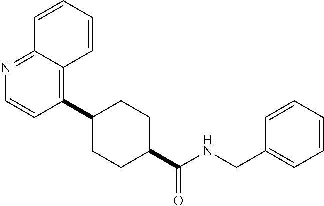 [SMILES: O=C(NCc1ccccc1)[C@H]1CC[C@@H](c2ccnc3ccccc32)CC1]

The title compound was prepared from 2-(cis-4-(quinolin-4-yl)cyclohexyl)acetic acid and phenylmethanamine according to the procedure described for the synthesis of 2-methyl-2-phenyl-N-(cis-4-(quinolin-4-yl)cyclohexyl)propanamide. ¹H NMR (400 MHz, DMSO) δ 8.82 (d, J=4.5 Hz, 1H), 8.36-8.28 (m, 1H), 8.23 (d, J=8.4 Hz, 1H), 8.02 (d, J=8.3 Hz, 1H), 7.80-7.70 (m, 1H), 7.67-7.58 (m, 1H), 7.38-7.20 (m, 6H), 4.32 (d, J=5.9 Hz, 2H), 3.49-3.40 (m, 1H), 2.70-2.62 (m, 1H), 2.15 (d, J=12.9 Hz, 2H), 1.94-1.70 (m, 6H). LCMS (ESI) m/z calcd for C₂₃H₂₄N₂O: 344.19. Found: 345.32 (M+1)⁺.

### IDO1 PBMC RapidFire MS Assay

Compounds of the present invention were tested via high-throughput cellular assays utilizing detection of kynurenine via mass spectrometry and cytotoxicity as end-points. For the mass spectrometry and cytotoxicity assays, human peripheral blood mononuclear cells (PBMC) (PB003F; AllCells®, Alameda, Calif.) were stimulated with human interferon-γ (IFN-γ) (Sigma-Aldrich Corporation, St. Louis, Mo.) and lipopolysaccharide from Salmonella minnesota (LPS) (Invivogen, San Diego, Calif.) to induce the expression of indoleamine 2, 3-dioxygenase (IDO1). Compounds with IDO1 inhibitory properties decreased the amount of kynurenine produced by the cells via the tryptophan catabolic pathway. Cellular toxicity due to the effect of compound treatment was measured using CellTiter-Glo® reagent (CTG) (Promega Corporation, Madison, Wis.), which is based on luminescent detection of ATP, an indicator of metabolically active cells.

In preparation for the assays, test compounds were serially diluted 3-fold in DMSO from a typical top concentration of 1mM or 5 mM and plated at 0.5 μL in 384-well, polystyrene, clear bottom, tissue culture treated plates with lids (Greiner Bio-One, Kremsmunster, Austria) to generate 11-point dose response curves. Low control wells (0% kynurenine or 100% cytotoxicity) contained either 0.5 μL of DMSO in the presence of unstimulated (-IFN-γ/-LPS) PBMCs for the mass spectrometry assay or 0.5 μL of DMSO in the absence of cells for the cytotoxicity assay, and high control wells (100% kynurenine or 0% cytotoxicity) contained 0.5 μL of DMSO in the presence of stimulated (+IFN-γ/+LPS) PBMCs for both the mass spectrometry and cytotoxicity assays.

Frozen stocks of PBMCs were washed and recovered in RPMI 1640 medium (Thermo Fisher Scientific, Inc., Waltham, Mass.) supplemented with 10% v/v heat-inactivated fetal bovine serum (FBS) (Thermo Fisher Scientific, Inc., Waltham, Mass.), and 1× penicillin-streptomycin antibiotic solution (Thermo Fisher Scientific, Inc., Waltham, Mass.). The cells were diluted to 1,000,000 cells/mL in the supplemented RPMI 1640 medium. 50 μL of either the cell suspension, for the mass spectrometry assay, or medium alone, for the cytotoxicity assay, were added to the low control wells, on the previously prepared 384-well compound plates, resulting in 50,000 cells/well or 0 cells/well respectively. IFN- y and LPS were added to the remaining cell suspension at final concentrations of 100 ng/ml and 50 ng/ml respectively, and 50 μL of the stimulated cells were added to all remaining wells on the 384-well compound plates. The plates, with lids, were then placed in a 37° C, 5% CO2 humidified incubator for 2 days.

Following incubation, the 384-well plates were removed from the incubator and allowed to equilibrate to room temperature for 30 minutes. For the cytotoxicity assay, CellTiter-Glo® was prepared according to the manufacturer's instructions, and 40 μL were added to each plate well. After a twenty minute incubation at room temperature, luminescence was read on an EnVision® Multilabel Reader (Perkin Elmer Inc., Waltham, Mass.). For the mass spectrometry assay, 10 μL of supernatant from each well of the compound-treated plates were added to 40 μL of acetonitrile, containing 10 μM of an internal standard for normalization, in 384-well, polypropylene, V-bottom plates (Greiner Bio-One, Kremsmunster, Austria) to extract the organic analytes. Following centrifugation at 2000 rpm for 10 minutes, 10 μL from each well of the acetonitrile extraction plates were added to 90 μL of sterile, distilled H2O in 384-well, polypropylene, V-bottom plates for analysis of kynurenine and the internal standard on the RapidFire 300 (Agilent Technologies, Santa Clara, Calif.) and 4000 QTRAP MS (SCIEX, Framingham, Mass.). MS data were integrated using Agilent Technologies' RapidFire Integrator software, and data were normalized for analysis as a ratio of kynurenine to the internal standard.

The data for dose responses in the mass spectrometry assay were plotted as % IDO1 inhibition versus compound concentration following normalization using the formula 100-(100*((U-C2)/(C1-C2))), where U was the unknown value, C1 was the average of the high (100% kynurenine; 0% inhibition) control wells and C2 was the average of the low (0% kynurenine; 100% inhibition) control wells. The data for dose responses in the cytotoxicity assay were plotted as % cytotoxicity versus compound concentration following normalization using the formula 100-(100*((U-C2)/(C1-C2))), where U was the unknown value, C1 was the average of the high (0% cytotoxicity) control wells and C2 was the average of the low (100% cytotoxicity) control wells. Curve fitting was performed with the equation y=A+((B-A)/(1+(10×/10C)D)), where A was the minimum response, B was the maximum response, C was the log(XC50) and D was the Hill slope. The results for each test compound were recorded as pIC50 values for the mass spectrometry assay and as pCC50 values for the cytoxicity assay (-C in the above equation).

| | PBMC | PBMC TOX |
|---|---|---|
| Example | pIC₅₀ | pIC₅₀ |
| 1 | 5.7 | <5 |
| 2 | 5.4 | <5 |
| 3 | 7.7 | <5 |
| 4 | 6.9 | <5 |
| 5 | 5.6 | <5 |
| 6 | 6.8 | <5 |
| 7 | 6.8 | <5 |
| 8 | 5.5 | <5 |
| 9 | <5.5 | <5 |
| 10 | 5.4 | <5 |
| 11 | <5 | <5 |
| 12 | <5 | <5 |
| 13 | <5 | <5 |
| 14 | <5 | <5 |
| 15 | <5 | <5 |
| 16 | <5 | <5 |
| 17 | 7 | <5 |
| 18 | 5.8 | <5 |
| 19 | 8.3 | <5 |
| 20 | 6.8 | <5 |
| 21 | 7.3 | <5 |
| 22 | 5.4 | <5 |
| 23 | <5 | <5 |
| 24 | 5.5 | <5 |
| 25 | 5.7 | <5 |
| 26 | 5.4 | <5 |
| 27 | 5.8 | <5 |
| 28 | <5 | <5 |
| 29 | 5.7 | <5 |
| 30 | 5.9 | <5 |
| 31 | 7.1 | <5 |
| 32 | 7.8 | <5 |
| 33 | 7.1 | <5 |
| 34 | 6.3 | <5 |
| 35 | 6.8 | <5 |
| 36 | 5.5 | <5 |
| 37 | 5.2 | <5 |
| 38 | 5.7 | <5 |
| 39 | 5.6 | <5 |
| 40 | 5.6 | <5 |
| 41 | 6.5 | <5 |
| 42 | 5.4 | <5 |
| 43 | <5 | <5 |
| 44 | 6.1 | <5 |
| 45 | 5 | <5 |
| 46 | 5.9 | <5 |

1. A compound of Formula I 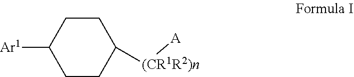
or a pharmaceutically acceptable salt thereof wherein:
AO is C₅₋₁₂aryl, or 5-12 membered heteroaryl, wherein aryl and heteroaryl include bicycles and heteroaryl contains 1-3 hetero atoms selected from O, S, and N, and wherein Ar¹ may optionally be substituted with 1-2 substituents independently selected from halogen, OH, C₁₋₃alkyl, OC₁₋₃alkyl, C₁₋₃fluoroalkyl, CN, and NH₂;
R¹ and R² are independently H or C₁₋₄alkyl;
n is 1 or 0;
A is —C(O)NR³R⁴—, —NR⁴C(O)R³—, —NR⁴C(O)C(R⁷)(R⁸)R³—, or Ar²-R⁵, wherein Ar² is C₅₋₁₂aryl, or 5-12 membered heteroaryl, wherein aryl and heteroaryl include bicycles and heteroaryl contains 1-3 hetero atoms selected from O, S, and N, and wherein Ar² may optionally be substituted with a substituent selected from halogen, OH, C₁₋₃alkyl, OC₁₋₃alkyl, C₁₋₃fluoroalkyl, CN, and NH₂;
R⁴, R⁷, and R⁸ are independently H or C₁₋₆alkyl;
R⁵ is H, Ci-6alkyl, C₅₋₇aryl, optionally substituted with a substituent selected from the group consisting of halogen, C₁₋₄alkyl, hydroxyl, —C(O)CH₃, C(O)OCH₃, and C(O)NH₂.
R³ is C₁₋₁₀alkyl, C₃₋₈cycloalkyl, or C₅₋₇aryl wherein R³ is optionally substituted with a substituent selected from the group consisting of halogen, C₁₋₄alkyl, hydroxyl, —C(O)CH₃, C(O)OCH₃, and C(O)NH₂.

2. A compound or salt according to claim 1 wherein Ar¹ is quinoline, isoquinoline, quinazoline, isoquinolone, quinazolone, naphthyridine, naphthalene, or indole, and may optionally be substituted with a substituent selected from halogen, OH, C₁₋₃alkyl, OC₁₋₃alkyl, C₁₋₃fluoroalkyl, CN, and NH₂.

3. A compound or salt according to claim 2 wherein AO is quinoline optionally substituted with a halogen.

4. A compound or salt according to claim 1 wherein R¹ and R² are independently H or methyl.

5. A compound or salt according to claim 1 any of claims 1 wherein Ar² is unsubstituted benzimidazole, 7-chloro-benzimidazole, oxazole, imidazole, 1,2,4-triazole, benzoxazolone, or benzoimidazolone.

6. A compound or salt according to claim 5 wherein Ar² is u unsubstituted benzimidazole or imidazole.

7. A compound or salt according to claim 1 wherein R⁵ is H, C₁₋₆alkyl, or phenyl optionally substituted with a halogen.

8. A compound or salt according to claim 1 wherein R³ is C₁₋₁₀alkyl, C₅₋₇cycloalkyl, or phenyl wherein R³ is optionally substituted with a substituent selected from the group consisting of halogen, C₁₋₃alkyl, hydroxyl, and C(O)NH₂.

9. A pharmaceutical composition comprising a compound or salt according to claim 1.

10. A method of treating a disease or condition that would benefit from inhibition of IDO 1 comprising the step of administration of a composition according to claim 9.

11. The method of claim 10 wherein in said disease or condition, biomarkers of IDO activity are elevated.

12. The method of claim 11 wherein said biomarkers are plasma kynurenine or the plasma kynurenine/tryptophan ratio.

13. The method of claim 10 wherein said disease or condition is chronic viral infections; chronic bacterial infections; cancer; sepsis; or a neurological disorder.

14. The method of claim 13 wherein said chronic viral infections are those involving HIV, HBV, or HCV; said chronic bacterial infections are tuberculosis or prosthetic joint infection; and said neurological disorders are major depressive disorder, Huntington's disease, or Parkinson's disease.

15. The method of claim 14 wherein said disease or condition is inflammation associated with HIV infection; chronic viral infections involving hepatitis B virus or hepatitis C virus; cancer; or sepsis.

16-17. (canceled)
